(19) United States
(12) Patent Application Publication
(10) Pub. No.: US 2006/0269550 A1
(43) Pub. Date: Nov. 30, 2006

(54) Anti-ghrelin fab antibodies

(76) Inventor: Mark Louis Heiman, Indianapolis, IN (US)

Correspondence Address: ELI LILLY & COMPANY, PATENT DIVISION, P.O. BOX 6288, INDIANAPOLIS, IN, 46206-6288, US

(21) Appl. No.: 10/558,604

(22) PCT Filed: May 28, 2004
(86) PCT No.: PCT/US04/14537

§ 371 Date: Nov. 29, 2005

Related U.S. Application Data
(60) Provisional application No. 60/475,708, filed on Jun. 4, 2003.
(60) Provisional application No. 60/491,352, filed on Jul. 31, 2003.
(60) Provisional application No. 60/501,465, filed on Sep. 9, 2003.

(51) Int. Cl. A61K 39/395; C07H 21/04; C12P 21/06; C07K 16/26; C12N 5/06

(52) U.S. Cl. 424/145.1; 435/69.1; 435/326; 435/320.1; 530/388.24; 536/23.53

(57) ABSTRACT

Monoclonal antibodies and antigen-binding portions thereof that preferentially bind acetylated human ghrelin with respect to unacylated human ghrelin are disclosed. These molecules have high affinity for acylated human ghrelin, a slow off rate for acylated human ghrelin dissociation, and neutralize an acylated human ghrelin activity. These antibodies or antigen-binding portions thereof are useful for neutralizing ghrelin activity, e.g., in a human subject suffering from a disorder in which ghrelin activity is detrimental.

### FIELD OF THE INVENTION

The present invention is in the field of medicine, particularly in the field of monoclonal antibodies against human ghrelin. More specifically the invention relates to monoclonal antibodies that preferentially bind acylated human ghrelin with respect to unacylated human ghrelin. The products are useful for treatment of obesity and obesity-related disorders including non-insulin dependent diabetes mellitus, Prader-Willi syndrome, hyperphagia, impaired satiety, eating disorders, gastric motility disorders, cardiovascular disease and cancer in mammals.

### BACKGROUND OF THE INVENTION

Obesity is a complex, chronic disease characterized by excessive accumulation of body fat and has a strong familial component. Obesity is generally the result of a combination of factors including genetic factors. Obesity increases the risk of illness from about 30 serious medical conditions including osteoarthritis, Type II diabetes, hypertension, cancer and cardiovascular disease, and is associated with increases in deaths from all causes. Additionally, obesity is associated with depression and can further affect the quality of life through limited mobility and decreased physical endurance.

There are presently limited treatments for obesity. Current treatment options to manage weight include dietary therapy, increased activity and behavior therapy. Unfortunately, these treatments are largely unsuccessful with a failure rate reaching 95%. This failure may be due to the fact that the condition is strongly associated with genetically inherited factors that contribute to increased appetite, preference for highly caloric foods, reduced physical activity and increased lipogenic metabolism. This indicates that people inheriting these genetic traits are prone to becoming obese regardless of their efforts to combat the condition. Gastric bypass surgery is available to a limited number of obese persons and drug therapy options are few and of limited utility.

Ghrelin is a recently identified hormone that, when acylated, binds the growth hormone secretagogue receptor (GHS-R1a) resulting in release of growth hormone. (Kojima, et al., Nature 402: 656-660, 1999). Additionally, ghrelin has been demonstrated to lead to fat deposition when administered to mice (Tschop, M. et al., Nature 407: 908-913, 2000). Ghrelin is synthesized primarily in the stomach and its levels increase during food deprivation in animals (Kojima, et al., Nature 402: 656-660, 1999) and peak prior to eating in humans (Cummings, et al., NEJM, 346:1623-1630, 2002). Recently it has been shown that persons who underwent gastric bypass surgery and lost up to 36% of their body weight had greatly reduced ghrelin levels, with loss of pre-meal peaks in ghrelin secretion. Persons with Prader-Willi syndrome, a genetic disorder that causes severe obesity with uncontrollable appetite, have extremely high levels of ghrelin. (Cummings, et al., NEJM, 346:1623-1630, 2002). These observations indicate that ghrelin plays a key role in motivating feeding.

International patent publication number WO 01/87335 teaches the use of agents which specifically bind ghrelin, including anti-ghrelin antibodies, for the treatment of obesity.

International patent publication number WO 01/07475 (EP 1197496) teaches the ghrelin amino acid sequence of various species including human and discloses that ghrelin is acylated with O-n-octanoic acid at the third amino acid from the amino terminus, which is serine in human ghrelin. WO 01/07475 also teaches that the amino terminal four amino acids of ghrelin are essential for ghrelin's calcium-releasing activity. The application further teaches antibodies directed against fatty acid-modified peptides of ghrelin, which peptides induce signal transduction, and the use of such antibodies for assaying or detecting ghrelin.

Murakami, N. et al., administered to obese rats by intracerebroventricular injection a polyclonal anti-ghrelin antibody raised against the acylated N-terminal 11 amino acids of rat ghrelin. The authors were able to demonstrate a subsequent decrease in both food intake and body weight by the rats (J. Endocrinology 174:283-288, 2002).

There is a tremendous therapeutic need for a means to treat obesity and obesity-related disorders. Due to its potential role in inducing feeding, ghrelin is a desirable target for therapeutic intervention. In particular, a monoclonal antibody against ghrelin that preferentially binds the acylated form of ghrelin with respect to the unacylated form of ghrelin may provide such a therapy. Of particular importance therapeutically is a chimeric form or humanized form of such a monoclonal antibody. Additionally, ghrelin is highly conserved in sequence and in function across species; therefore, not only may such an antibody be useful for the treatment of such disorders in humans, but also in other mammals including, e.g., domestic animals (e.g., canine and feline) and food-source animals (e.g., bovine, porcine and ovine). Such an anti-ghrelin antibody may be useful for the treatment of obesity and related disorders including, for example, Type II non-insulin dependent diabetes mellitus (NIDDM), Prader-Willi syndrome, hyperphagia and impaired satiety, eating disorders in mammals. The antibodies of the invention may further be used for treatment of gastric motility disorders, cardiovascular disease and cancer in mammals.

### BRIEF SUMMARY OF THE INVENTION

Monoclonal antibodies against human ghrelin (“hGhrelin”) that preferentially bind the acylated form of hGhrelin with respect to the des-acyl, (unacylated) form of hGhrelin are described in the present invention. Such antibodies are also referred to herein as “anti-hGhrelin monoclonal antibodies.” The antibodies of the invention include murine monoclonal antibodies, chimeric monoclonal antibodies and “humanized” monoclonal antibodies or “deimmunized” monoclonal antibodies. Preferably the antibodies exist in a homogeneous population.

The antibodies of the invention are characterized by high affinity binding to acylated hGhrelin but not to des-acyl hGhrelin, and the capacity to disrupt or antagonize at least one in vitro or in vivo activity associated with acylated hGhrelin.

The invention provides an anti-hGhrelin monoclonal antibody comprising at least 1, 2, 3, 4 or 5 peptides from peptides with a sequence selected from the group consisting of SEQ ID NOs: 5, 8, 10, 19, 21 and 25. One embodiment provides an anti-hGhrelin monoclonal antibody comprising the 6 peptides with the sequences as shown in SEQ ID NOs: 5, 8, 10, 19, 21 and 25. In a preferred embodiment, an anti-hGhrelin monoclonal antibody of the invention comprises a light chain variable region (LCVR) comprising a peptide with the sequence shown in SEQ ID NO: 3 or comprises a heavy chain variable region (HCVR) comprising a peptide with the sequence shown in SEQ ID NO: 16. In a more preferred embodiment, an anti-hGhrelin monoclonal antibody of the invention comprises a LCVR comprising a peptide with the sequence shown in SEQ ID NO: 3 and further comprises a HCVR comprising a peptide with the sequence shown in SEQ ID NO: 16. In another preferred embodiment, an anti-hGhrelin monoclonal antibody of the invention comprises at least 1, 2, 3, 4 or 5 peptides (or all six peptides) with a sequence selected from the group consisting of SEQ ID NOs: 5, 8, 10, 19, 21 and 25 wherein said peptide exists in said antibody at the same CDR position as shown in Tables 1, 6 or 7 herein.

Preferably the LCVR CDR1 of an anti-hGhrelin monoclonal antibody of the invention comprises a peptide with the sequence shown in SEQ ID NO: 5. Preferably the LCVR CDR2 of an anti-hGhrelin monoclonal antibody of the invention comprises a peptide with the sequence shown in SEQ ID NO: 8. Preferably the LCVR CDR3 of an anti-hGhrelin monoclonal antibody of the invention comprises a peptide with the sequence shown in SEQ ID NO: 10. Preferably the HCVR CDR1 of an anti-hGhrelin monoclonal antibody of the invention comprises a peptide with the sequence shown in SEQ ID NO: 19. Preferably the HCVR CDR2 of an anti-hGhrelin monoclonal antibody of the invention comprises a peptide with the sequence shown in SEQ ID NO: 21. And preferably the HCVR CDR3 of an anti-hGhrelin monoclonal antibody of the invention comprises a peptide with the sequence shown in SEQ ID NO: 25.

An anti-hGhrelin monoclonal antibody of the invention may further comprise a heavy chain constant region selected from the group consisting of IgG1, IgG2, IgG3, IgG4, IgA, IgE, IgM and IgD. Preferably the heavy chain constant region is selected from the group consisting of IgG1, IgG2, IgG3 or IgG4. An anti-hGhrelin monoclonal antibody of the invention may further comprise a kappa or lambda light chain constant region.

An anti-hGhrelin monoclonal antibody of the invention may comprise or consist of an intact antibody, a substantially intact antibody, a Fab fragment, a F(ab′)2 fragment or a single chain Fv fragment.

An anti-hGhrelin monoclonal antibody of the invention may comprise 1, 2, 3, 4, 5 or 6 peptides selected from peptides with a sequence selected from the group consisting of SEQ ID Nos: 5, 8, 10, 19, 21 and 25 in which said peptide has 2 or 1 conservative amino acid substitutions and/or terminal deletions with respect to the sequence shown in said SEQ ID Nos: 5, 8, 10, 19, 21 and 25.

In a preferred embodiment, an anti-hGhrelin monoclonal antibody of the invention is a chimeric antibody. In a more preferred embodiment, an anti-hGhrelin monoclonal antibody of the invention is humanized. In another preferred embodiment an anti-hGhrelin monoclonal antibody of the invention is deimmunized.

In another embodiment, the invention provides an isolated nucleic acid molecule that encodes an anti-hGhrelin monoclonal antibody of the invention. A preferred isolated nucleic acid molecule of the invention encodes an anti-hGhrelin monoclonal antibody of the invention and comprises a polynucleotide selected from the group consisting of polynucleotides comprising a sequence as shown in SEQ ID NOs: 6, 7, 9, 11, 12, 13, 14, 15, 17, 18, 20, 22, 23 and 24. Preferably said nucleic acid molecule is operably linked to a promoter.

In another embodiment, the invention provides a vector, preferably a recombinant expression vector, comprising a polynucleotide encoding an anti-hGhrelin monoclonal antibody of the invention. In another embodiment, the vector comprises a nucleic acid molecule of the invention that encodes an anti-hGhrelin monoclonal antibody of the invention. In another embodiment, the vector comprises a polynucleotide with a sequence selected from the group consisting of SEQ ID NOs: 6, 7,9, 11, 12, 13, 14, 15, 17, 18,20, 22, 23 and 24.

In another embodiment, the invention provides a host cell into which a vector of the invention has been introduced. Preferably the host cell is a CHO cell, a COS. cell or a NS0 cell or a derivative thereof or a yeast cell.

In another embodiment, the invention provides a method of synthesizing an anti-hGhrelin monoclonal antibody of the invention comprising culturing a host cell of the invention in culture media such that an anti-hGhrelin monoclonal antibody of the invention is expressed in the cell. Preferably the antibody is purified from the cell or from the culture media in which said cell is grown.

Various forms of the antibodies of the invention are contemplated herein. For example, an anti-hGhrelin monoclonal antibody of the invention may be a full length antibody (e.g., having a human or murine immunoglobulin constant region) or an antibody fragment(e.g., a F(ab′)2). It is understood that all such forms of the antibodies are encompassed herein within the term “antibody.” Furthermore, the antibody may be labeled with a detectable label, immobilized on a solid phase and/or conjugated with a heterologous compound (e.g., an enzyme or toxin) according to methods known in the art.

Diagnostic uses for antibodies of the invention are contemplated. In one diagnostic application, the invention provides a method for determining the presence of ghrelin peptide comprising exposing a test sample suspected of containing the ghrelin peptide to an antibody of the invention and determining specific binding of the antibody to the sample. An anti-hGhrelin antibody of the invention may be used to determine the levels of acylated ghrelin in test samples by comparing test sample values to a standard curve generated by binding said antibody to samples with known amounts of acylated ghrelin. For diagnostic use, the invention provides a kit comprising an antibody of the invention and instructions for using the antibody to detect acylated ghrelin protein in a test sample.

In another embodiment, the invention provides a pharmaceutical composition comprising an anti-hGhrelin monoclonal antibody of the invention. The pharmaceutical composition of the invention may further comprise a pharmaceutically acceptable carrier. In said pharmaceutical composition, the anti-hGhrelin monoclonal antibody of the invention is the active ingredient. Preferably the pharmaceutical composition comprises a homogeneous population of an anti-hGhrelin monoclonal antibody. The composition for therapeutic use is sterile and may be lyophilized.

The invention further provides a method of inhibiting ghrelin activity in a mammal in need thereof by administering a therapeutically effective amount, or prophylactically effective amount, of an anti-hGhrelin monoclonal antibody of the invention to said mammal. The invention further provides a method of treating or preventing a disease or disorder ameliorated by the inhibition of binding of ghrelin to its receptor or inhibition of signal transduction resulting from the binding of ghrelin to its receptor which comprises administering to a patient (e.g., a human) in need of such treatment or prevention a therapeutically or prophylactically effective amount of an antibody of the invention. Such diseases or disorders include, but are not limited to, obesity, NIDDM, Prader-Willi syndrome, eating disorders, hyperphagia, impaired satiety, anxiety, gastric motility disorders (including e.g., irritable bowel syndrome and functional dyspepsia), cancer and cardiovascular disorders. The invention further provides a method for treating or preventing obesity and disorders related to obesity including for example, NIDDM, Prader-Willi syndrome, hyperphagia and impaired satiety in a mammal in need thereof by administering a therapeutically effective amount or prophylactically effective amount of an anti-hGhrelin monoclonal antibody of the invention. Preferably the mammal is a human, canine, feline, equine, ovine, porcine or bovine, most preferably a human.

The invention further embodies an anti-hGhrelin monoclonal antibody of the invention for use in the manufacture of a medicament for administration to a mammal for the treatment or prevention of obesity and disorders related to obesity (including for example, NIDDM, Prader-Willi syndrome, hyperphagia and impaired satiety), anxiety, gastric motility disorder (including e.g., irritable bowel syndrome and functional dyspepsia), cancer and cardiovascular disease in a mammal in need thereof by administering a therapeutically effective amount or prophylactically effective amount of an anti-hGhrelin monoclonal antibody of the invention. Preferably the mammal is a human, canine, feline, equine, ovine, porcine or bovine, most preferably a human.

The invention embodies an article of manufacture comprising a packaging material and an antibody of the invention contained within said packaging material and wherein the packaging material comprises a package insert which indicates that the antibody neutralizes a ghrelin activity by preferentially binding acylated ghrelin with respect to unacylated ghrelin.

TABLE 1
CDR Sequences of 1111,2211,2291,2891,1481

| | | | |
|---|---|---|---|
| Domain | CDR1 | CDR2 | CDR3 |

| | | | |
|---|---|---|---|
| 1111 VH | GYIFTGYWIE | EILPGSGSTNYNEKFKG | YPQFRLRRERIAY |
| | (SEQ ID NO: 19) | (SEQ ID NO: 21) | (SEQ ID NO: 25) |
| 2211 VH | GYTFTGYWIE | EILPGSGSTNYNEKFKG | YPQFGLRRERIAY |
| | (SEQ ID NO: 19) | (SEQ ID NO: 21) | (SEQ ID NO: 25) |
| 2291 VH | GYTFTGYWIE | EILPGSGSTNYNEKFKG | YPQFRLRRERIAY |
| | (SEQ ID NO: 19) | (SEQ ID NO: 21) | (SEQ ID NO: 25) |
| 2891 VH | GYTFTGYWIE | EILPGSGSTNYNEKFKG | YPQFRLRRERIAY |
| | (SEQ ID NO: 19) | (SEQ ID NO: 21) | (SEQ ID NO: 25) |
| 1481 VH | GYTFTGYWIE | EILPGSGSTNYNEKFKG | YPQFRLRRGRIAY |
| | (SEQ ID NO: 19) | (SEQ ID NO: 21) | (SEQ ID NO: 25) |
| 1111 VL | RASKSVSTSGYSYSYMH | LASNLES | QHSRELPYTF |
| | (SEQ ID NO: 5) | (SEQ ID NO: 8) | (SEQ ID NO: 10) |
| 2211 VL | RASKSVSTSGYSYSYMH | LASNLES | QHSRELPYTF |
| | (SEQ ID NO: 5) | (SEQ ID NO: 8) | (SEQ ID NO: 10) |
| 2291 VL | RASKSVSTSGYSYSYMH | LASNLEP | QHSRELPYTF |
| | (SEQ ID NO: 5) | (SEQ ID NO: 8) | (SEQ ID NO: 10) |
| 2891 VL | RASKSVSTSGYSYSYMH | LASNLES | QHSRELPYTF |
| | (SEQ ID NO: 5) | (SEQ ID NO: 8) | (SEQ ID NO: 10) |
| 1481 VL | RASKSVSTSGYSYSYMH | LASNLES | QHSRELPYTF |
| | (SEQ ID NO: 5) | (SEQ ID NO: 8) | (SEQ ID NO: 10) |

### BRIEF DESCRIPTION OF THE DRAWINGS

FIG. 1 shows inhibition of ghrelin agonists by Fabs 1111, 2211, 2291 and 2891 as described in Example 3.

FIG. 2 shows FLPR assay results for Fabs 1111 and 2291 as described in Example 3.

FIG. 3 shows the structure of acylated human ghrelin (hGhrelin) and the structure of hGhrelin-Dap3-octanamide

### DETAILED DESCRIPTION OF THE INVENTION

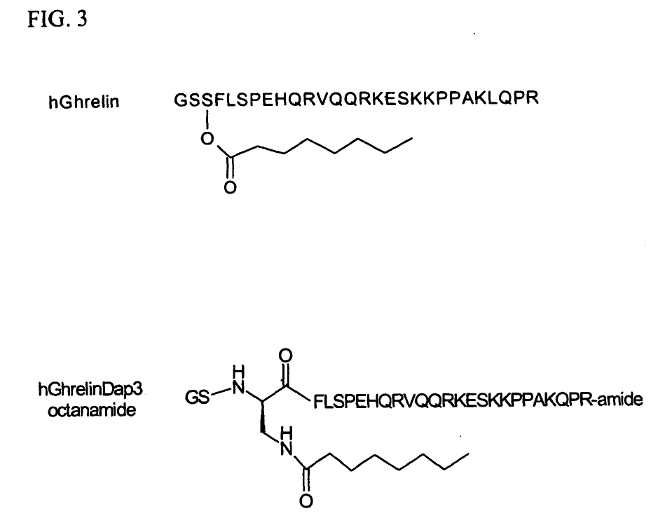

Ghrelin was identified as the endogenous ligand of the growth hormone secretagogue receptor (GHS-R1a) (Kojima, M. et al. Nature 402:656-660, 1999). It is secreted from multiple organs of the body but primarily from the stomach. The predominant form of ghrelin present in humans is a 28 amino acid peptide with an n-octanoyl modification at the serine amino acid located at position 3 (FIG. 3). The unacylated, or “des-acyl” form of ghrelin does not bind GSH-R1a. A monoclonal antibody, as described in the present invention, specifically directed against the acylated form of ghrelin may have a greater likelihood of being an effective therapeutic than a monoclonal antibody that reacts with both the acylated and unacylated form of ghrelin due to the high percentage of ghrelin which exists in the unacylated form in the body, but which is not able to bind GHS-R1a as does the acylated form of ghrelin nor trigger subsequent biochemical processes as does the acylated form of ghrelin.

Recently peptides with various modifications of the predominant form of ghrelin have been identified in human stomach (Hosoda, H. et al., J. Biol. Chem. 278:64-70, 2003). These minor forms include a 27 amino acid ghrelin peptide lacking the C-terminal Arg of SEQ ID NO: 26 and ghrelin peptides decanoylated or decenoylated at position 3. It is contemplated that the antibodies of the present invention preferentially bind both the 28 and 27 amino acid forms of ghrelin (or even shorter forms when C-terminal deleted) when acylated at position 3 (whether it be with an n-octanoyl, decanoyl or decenoyl group or other fatty acid) in relation to des-acyl ghrelin.

The predominant form of acylated ghrelin is referred to herein as “acylated ghrelin,” or “acylated hGhrelin” when referring to human ghrelin. When referring to the unacylated form of ghrelin, the term “des-acyl ghrelin” or “des-acyl hGhrelin” is used herein. When “ghrelin” or “hGhrelin” is used without referring to a form of acylation; the acylated form is implied. The anti-hGhrelin monoclonal antibodies of the invention (“antibodies of the invention”) preferentially bind acylated hGhrelin with respect to des-acyl hGhrelin; the epitope to which they bind has been localized to amino acids 1-8 of acylated hGhrelin (see Examples 2 and 3 herein) and the sequence of this fragment is identical in the ghrelin peptide of many other species including murine, rat, canine, feline, bovine, ovine and porcine. Therefore, an antibody of the invention is contemplated to preferentially bind acylated hGhrelin with respect to des-acyl hGhrelin as well as acylated ghrelin with respect to des-acyl ghrelin in other species with the same or substantially similar sequence at amino acids 1-8 of ghrelin (i.e., NH2-GSSFLSPE) acylated at position 3 from the amino terminus.

A full-length antibody as it exists naturally is-an immunoglobulin molecule comprised of four peptide chains, two heavy (H) chains (about 50-70 kDa when full length) and two light (L) chains (about 25 kDa when full length) inter-connected by disulfide bonds. The amino terminal portion of each chain includes a variable region of about 100-110 or more amino acids primarily responsible for antigen recognition. The carboxy terminal portion of each chain defines a constant region primarily responsible for effector function.

Light chains are classified as kappa and lambda and characterized by a particular constant region. Heavy chains are classified as gamma, mu, alpha, delta, or epsilon, and define the antibody's isotype as IgG, IgM, IgA, IgD, and IgE, respectively. Each heavy chain type is characterized by a particular constant region. Each heavy chain is comprised of a heavy chain variable region (herein “HCVR”) and a heavy chain constant region. The heavy chain constant region is comprised of three domains (CH)1, CH2, and CH3) for IgG, IgD, and IgA; and 4 domains (CH1, CH2, CH3, and CH4) for IgM and IgE. Each light chain is comprised of a light chain variable region (herein “LCVR”) and a light chain constant region. The light chain constant region is comprised of one domain, CL. The HCVR and LCVR regions can be further subdivided into regions of hypervariability, termed complementarity determining regions (CDRs), interspersed with regions that are more conserved, termed framework regions (FR). Each HCVR and LCVR is composed of three CDRs and four FRs, arranged from amino-terminus to carboxy-terminus in the following order: FR1, CDR1, FR2, CDR2, FR3, CDR3, FR4. The assignment of amino acids to each domain is in accordance with well-known conventions (Kabat, “Sequences of Proteins of Immunological Interest,” National Institutes of Health, Bethesda, Md. (1987 and 1991)). The functional ability of the antibody to bind a particular antigen is determined collectively by the six CDRs. However, even a single variable domain comprising only three CDRs specific for an antigen may have the ability to recognize and bind antigen, although at a lower avidity than a complete antibody.

The term “antibody,” in reference to an anti-hGhrelin antibody or anti-ghrelin antibody of the invention as used herein, refers to a monoclonal antibody. A “monoclonal antibody” as used herein refers to a murine monoclonal antibody or a chimeric antibody or a humanized antibody. A monoclonal antibody can be an intact (complete) antibody, a substantially intact antibody, a portion of an antibody comprising an antigen-binding portion, a Fab fragment, Fab′ fragment or F(ab′)₂ fragment of a murine antibody or of a humanized antibody. Furthermore, a monoclonal antibody can be a single chain Fv fragment which may be produced by joining the DNA encoding the LCVR and HCVR with a linker sequence. (See, Pluckthun, The Pharmacology of Monoclonal Antibodies, vol. 113, Rosenburg and Moore eds., Springer-Verlag, New York, pp 269-315, 1994). It is understood that regardless of whether fragments are specified, the term “antibody” includes such fragments as well as single chain forms. As long as the protein retains the ability to specifically bind its intended target, it is included within the term “antibody.” Antibodies may or may not be glycosylated, though glycosylated antibodies are preferred.

A “monoclonal antibody” as used herein when referring to a population of antibodies, refers to a homogeneous antibody population (i.e., at least about 90%, 95%, 96%, more preferably at least about 97% or 98% or most preferably at least 99% of the antibodies in the population are identical (i.e., they would compete in an ELISA assay for the same antigen)). The monoclonal antibody may be expressed by a hybridoma, expressed recombinantly, or synthesized synthetically by means readily known in the art.

The term “humanized antibody,” as used herein, is an intact antibody, a substantially intact antibody, a portion of an antibody comprising an antigen-binding site, or a portion of an antibody comprising a Fab fragment, Fab′ fragment, F(ab′)2, or a single chain Fv fragment having CDRs that originate from a mouse monoclonal antibody or are otherwise non-human in origin and framework and constant region to the extent it is present (or a substantial portion thereof, i.e., at least about 90%, 92%, 94%, 96%, 98% or 99%) encoded by nucleic acid sequence information that occurs in the human germline immunoglobulin region or in recombined or mutated forms thereof whether or not said antibodies are produced in human cells. It is contemplated that in the process of creating a humanized antibody, the amino acid at either termini of a CDR may be substituted with an amino acid that occurs in the human germline for that segment of adjoining framework sequence. Preferably a therapeutic antibody of the invention would have sequence of the framework and/or constant region derived from the mammal in which it would be used as a therapeutic so as to decrease the possibility that the mammal would illicit an immune response against the therapeutic antibody.

The phrases “biological property” or “biological characteristic,” or the terms “activity” or “bioactivity,” in reference to an antibody of the present invention, are used interchangeably herein and include, but are not limited to, epitope affinity and specificity (e.g., anti-ghrelin monoclonal antibody binding to ghrelin), ability to antagonize an activity of the targeted peptide in vivo or in% vitro (e.g., ghrelin bioactivity), the in vivo stability of the antibody and the immunogenic properties of the antibody. Other identifiable biological properties or characteristics of an antibody recognized in the art include, for example, cross-reactivity, (i.e., with non-human homologs of the targeted peptide, or with other proteins or tissues, generally), and ability to preserve high expression levels of protein in mammalian cells. The aforementioned properties or characteristics can be observed or measured using art-recognized techniques including, but not limited to ELISA, competitive ELISA, BIAcore® surface plasmon resonance analysis, in vitro and in vivo neutralization assays (e.g., Examples 2, 3 and 4), and immunohistochemistry with tissue sections from different sources including human, primate, or any other source as the need may be.

The term “epitope” as used herein refers to a region of a protein molecule to which an antibody can bind. An “immunogenic epitope” is defined as the part of a protein that elicits an antibody response when the whole protein is the immunogen. The anti-hGhrelin monoclonal antibodies of the invention bind an epitope localized to within amino acids 1-8 of acylated human ghrelin.

The term “inhibit” or “inhibiting” means neutralizing, antagonizing, prohibiting, preventing, restraining, slowing, disrupting, stopping, or reversing progression or severity of that which is being inhibited, e.g., including, but not limited to an activity, a disease or condition.

The term “isolated” when used in relation to a nucleic acid or protein (e.g., an antibody), refers to a nucleic acid sequence or protein that is identified and separated from at least one contaminant (nucleic acid or protein, respectively) with which it is ordinarily associated in its natural source. Isolated nucleic acid or protein is present in a form or setting that is different from that in which it is found in nature. In contrast, non-isolated nucleic acids or proteins are found in the state they exist in nature. Preferably, an “isolated antibody” is an antibody that is substantially free of other antibodies having different antigenic specificities (e.g., pharmaceutical compositions of the invention comprise an isolated antibody that specifically binds ghrelin substantially free of antibodies that specifically bind antigens other than ghrelin peptide).

The terms “Kabat numbering” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody (Kabat, et al., Ann. NY Acad. Sci. 190:382-93 (1971); Kabat, et al., Sequences of Proteins of Immunological Interest, Fifth Edition, U.S. Department of Health and Human Services, NIH Publication No. 91-3242 (1991)).

A polynucleotide is “operably linked” when it is placed into a functional relationship with another polynucleotide. For example, a promoter or enhancer is operably linked to a coding sequence if it affects the transcription of the sequence.

Recombinant humanized antibodies may be subjected to in vitro mutagenesis using methods of routine use in the art and, thus, the framework region amino acid sequences of the HCVR and LCVR regions of the humanized recombinant antibodies are sequences that, while derived from those related to human germline HCVR and LCVR sequences, may not naturally exist within the human antibody germ line repertoire in vivo. It is contemplated that such amino acid sequences of the HCVR and LCVR framework regions of the humanized recombinant antibodies are at least 90%, 92%, 94%, 96%, 98% or most preferably at least 99% identical to a human genrline sequence.

The term “neutralizing” or “antagonizing” in reference to an anti-hGhrelin (or anti-ghrelin) monoclonal antibody of the invention or the phrase “antibody that antagonizes (neutralizes) ghrelin activity” or “antagonizes (neutralizes) ghrelin” is intended to refer to an antibody whose binding to or contact with hGhrelin results in inhibition of a biological activity induced by acylated human ghrelin. Inhibition of hGhrelin biological activity can be assessed by measuring one or more in vitro or in vivo indicators of hGhrelin biological activity including, but not limited to, induction of weight loss, altered feeding, or inhibition of receptor binding (see WO 01/87335 for exemplary receptor binding assay) or signal transduction in a ghrelin-receptor binding assay. Indicators of ghrelin biological activity can be assessed by one or more of the several in vitro or in vivo assays known in the art. Preferably, the ability of an anti-ghrelin antibody to neutralize or antagonize ghrelin activity is assessed by use of the FLPR assay as described in Example 3 herein or by weight loss when being tested in vivo.

The terms “individual, ” “subject,” and “patient,” used interchangeably herein, refer to a mammal, including, but not limited to, murines, simians, humans, mammalian farm animals, mammalian sport animals, and mammalian pets; preferably humans.

The term “K_{off},” as used herein, refers to the off rate constant for dissociation of an antibody from the antibody/antigen complex. The dissociation rate constant (K_{off}) of an anti-ghrelin monoclonal antibody can be determined by BIAcore® surface plasmon resonance as generally described in Example 4 herein. Generally, BIAcore® analysis measures real-time binding interactions between ligand (recombinant ghrelin peptide immobilized on a biosensor matrix) and analyte (antibodies in solution) by surface plasmon resonance (SPR) using the BIAcore system (Pharmacia Biosensor, Piscataway, N.J.). SPR can also be performed by immobilizing the analyte (antibodies on a biosensor matrix) and presenting the ligand in solution.

The term “K_{D},” as used herein, is refers to the equilibrium dissociation constant of a particular antibody-antigen interaction. For purposes of the present invention, K_{D} is determined as shown in Example 4. Antibodies with high avidity and/or high affinity binding with a particular epitope have a K_{D} of 10⁻⁷ M or less, preferably 10⁻⁸ M or less, more preferably 10⁻⁹ M or less.

The term “vector” includes a nucleic acid molecule capable of transporting another nucleic acid to which it has been linked including, but not limited to, plasmids and viral vectors. Certain vectors are capable of autonomous replication in a host cell into which they are introduced while other vectors can be integrated into the genome of a host cell upon introduction into the host cell, and thereby, are replicated along with the host genome. Moreover, certain vectors are capable of directing the expression of genes to which they are operably linked. Such vectors are referred to herein as “recombinant expression vectors” (or simply “expression vectors”) and exemplary vectors are well known in the art.

The term “host cell” includes an individual cell or cell culture that can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention may also be referred to as a “recombinant host cell”. Preferred host cells for use in the invention are CHO cells, NS0 cells, SP2/0 cells and COS cells.

The present invention relates to monoclonal antibodies that preferentially bind acylated hGhrelin in relation to des-acyl hGhrelin (see Examples 2 and 3 herein). Also disclosed are antibody heavy and light chain fragments that are highly specific for acylated hGhrelin in relation to des-acyl hGhrelin, and neutralize hGhrelin or a hGhrelin activity, preferably the binding of hGhrelin to GHS-R1a or the prompting of a signal transduction response by hGhrelin through this receptor. This high specificity for binding ghrelin enables the anti-hGhrelin monoclonal antibodies of the invention, (including antigen-binding portions thereof, and humanized monoclonal antibodies with like specificity), to be immunotherapeutic to ghrelin-associated diseases and disorders. The epitope to which the antibodies of the invention bind is located within the amino terminal eight amino acids of acylated hGhrelin.

As used herein, an “acylated human ghrelin peptide” is a peptide comprising or consisting of amino acids 1-8 of SEQ ID NO: 26 incrementally adding one amino acid as shown in SEQ ID NO: 26 to the C-terminal end of peptide 1-8 up to an amino acid with the sequence as shown in SEQ ID NO: 26 (for example, amino acids 1-9, 1-10, 1-11 up to 1-28 of SEQ ID NO: 26); each said peptide acylated at the third amino acid from the amino terminus, preferably acylated with an N-octanoyl, decanoyl or decenoyl group In a preferred embodiment, the invention provides an anti-hGhrelin monoclonal antibody that binds an acylated human ghrelin peptide with an equilibrium dissociation constant, K_{D}, of 2×10⁻⁷ M or less, more preferably 2×10⁻⁸ M or less and even more preferably 2×10⁻⁹ M or less (as determined by solid phase BIAcore® surface plasmon resonance at room temperature) and has the capacity to antagonize an activity of acylated human ghrelin peptide (See, e.g., Example 4 herein).

“Preferentially binds” as used herein in reference to an anti-ghrelin (or anti-hGhrelin) monoclonal antibody of the invention describes the ability of said antibody to bind an acylated human ghrelin peptide, as described above, at least 10, 20, 50, 70, 100, 200 or 500 fold greater than it binds an unacylated form of said peptide. This may be measured by any of a number of assays known in the art including, but not limited to, a competitive ELISA assay as described in Example 2 herein or a surface plasmon resonance assay as described in Example 4.

Another embodiment of the invention provides an anti-hGhrelin monoclonal antibody that inhibits an acylated hGhrelin-mediated activity as represented, e.g., by the FLPR assay described in Example 3 herein. Preferably, said acylated hGhrelin-mediated activity is inhibited with an ICSO of 40 nM or less, more preferably 10 nM or less, 5 nM or less, 4 nM or less, 3nM or less, most preferably 2 nM or less, or 1 nM or less for or an IC₅₀ of 0.2 nM or less.

The most preferred anti-hGhrelin monoclonal antibody of the present invention comprises the sequence as shown in SEQ ID NOs: 3 and 16, that referred to herein as 1111. Exemplary polynucleotide sequences encoding the LCVR and HCVR of Fab 1111 are shown in SEQ ID NO: 2 and SEQ ID NO: 12 respectively.

In another embodiment, a preferred anti-hGhrelin monoclonal antibody is that referred to herein as 2291. The 2291 antibody has LCVR and HCVR comprising a peptide with a sequence as shown in SEQ ID NO: 3 and SEQ ID NO: 16, respectively (see Tables 6 and 7 herein). Exemplary polynucleotide sequences encoding the LCVR and HCVR of 2291 are shown in SEQ ID NO: 1 and SEQ ID NO: 11 respectively.

The invention further provides an isolated anti-hGhrelin monoclonal antibody comprising at least one complementarity determining region (CDR) comprising a peptide with an amino acid sequence selected from the group consisting of SEQ ID NOs: 5, 8, 10, 19, 21 and 25. Preferably, the amino acid sequence as shown SEQ ID NO: 5, when it exists in an antibody of the invention, is located at CDR1, most preferably the CDR1 of LCVR. Preferably the amino acid sequence as shown in SEQ ID NO: 8, when it exists in an antibody of the invention, is located at CDR2, most preferably the CDR2 of LCVR. And, preferably the amino acid sequence as shown in SEQ ID NO: 10 when it exists in an antibody of the invention, is located at CDR3, most preferably CDR3 of LCVR. Preferably, the amino acid sequence as shown SEQ ID NO: 19, when it exists in an antibody of the invention, is located at CDR1, most preferably CDR1 of HCVR. Preferably the amino acid sequence as shown in SEQ ID NO: 21, when it exists in an antibody of the invention, is located at CDR2, most preferably CDR2 of HCVR. And, preferably the amino acid sequence as shown in SEQ ID NO: 25 when it exists in an antibody of the invention, is located at CDR3, most preferably CDR3 of HCVR.

In another embodiment, the present invention is also directed to cell lines that produce an anti-hGhrelin monoclonal antibody of the invention. Creation and isolation of cell lines producing a monoclonal antibody of the invention can be accomplished using routine screening techniques known in the art. Preferred cell lines include COS, CHO and NS0 (available from ATCC, American Type Culture Collection, Manassas, Va.).

A wide variety of host expression systems can be used to express an antibody of 10 the present invention including prokaryotic (bacterial) and eukaryotic expression systems (such as yeast, baculoviral, plant, mammalian and other animal cells, transgenic animals, and hybridoma cells), as well as phage display expression systems. An example of a suitable bacterial expression vector is pUC119 and a suitable eukaryotic expression vector is a modified pcDNA3.1 vector with a weakened DHFR selection system. Other antibody expression systems are also known in the art and are contemplated herein.

An antibody of the invention can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell. Preferably, the recombinant antibodies are secreted into the medium in which the host cells are cultured, from which the antibodies can be recovered or purified. Standard recombinant DNA methodologies are used to obtain antibody heavy and light chain genes, incorporate these genes into recombinant expression vectors, and introduce the vectors into host cells. Such standard recombinant DNA technologies are described, for example, in Sambrook, Fritsch, and Maniatis (Eds.), Molecular Cloning; A Laboratory Manual, Second Edition, Cold Spring Harbor, N.Y., (1989); Ausubel, et al (Eds.) Current Protocols in Molecular Biology, Greene Publishing Associates, (1989); and in U.S. Pat. No. 4,816,397.

An isolated DNA encoding a HCVR region can be converted to a full-length heavy chain gene by operably linking the HCVR-encoding DNA to another DNA molecule encoding heavy chain constant regions (CH1, CH2, and CH3). The sequences of human heavy chain constant region genes are known in the art. See, e.g., Kabat, et al., Sequences of Proteins of Immunological Interest, Fifth Edition, U.S. Department of Health and Human Services, NIH Publication No. 91-3242 (1991). DNA fragments encompassing these regions can be obtained by standard PCR amplification. The heavy chain constant region can be an IgG (further divided into isotypes IgG1, IgG2, IgG3 and IgG4), IgA, IgE, IgM or IgD constant region and any allotypic variant thereof as described in Kabat (supra), but most preferably is an IgG4 or an IgG1 constant region. Alternatively, the antigen binding portion can be a Fab fragment, a F(ab′)₂ fragment, or a single chain Fv fragment (scFv). For a Fab fragment heavy chain gene, the HCVR-encoding DNA can be operably linked to-another DNA molecule encoding only a heavy chain CH1 constant region.

An isolated DNA encoding a LCVR region can be converted to a full-length light chain gene (as well as a Fab light chain gene) by operably linking the LCVR-encoding DNA to another DNA molecule encoding a light chain constant region, CL. The sequences of human light chain constant region genes are known in the art. See, e.g., Kabat, supra. DNA fragments encompassing these regions can be obtained by standard PCR amplification. The light chain constant region can be a kappa or lambda constant region.

To create an scFv gene, the HCVR- and LCVR-encoding DNA fragments are operably linked to another fragment encoding a flexible linker, e.g. encoding the amino acid sequence (Gly₄-Ser)₃, such that the HCVR and LCVR sequences can be expressed as a contiguous single-chain protein, with the LCVR and HCVR regions joined by the flexible linker. See, e.g., Bird, et al., Science 242:423-6 (1988); Huston, et al., Proc. Natl. Acad. Sci. USA 85:5879-83 (1988); McCafferty, et al., Nature 348:552-4 (1990).

To express an antibody of the invention, a DNA encoding a partial or full-length light and/or heavy chain, obtained as described above, are inserted into an expression vector such that the gene is operably linked to transcriptional and translational control sequences. The expression vector and expression control sequences are chosen to be compatible with the expression host cell used. The antibody light chain gene and the antibody heavy chain gene can be inserted into separate vectors or, more typically, both genes are inserted into the same expression vector. The antibody genes are inserted into the expression vector by standard methods. Additionally, the recombinant expression vector can encode a signal peptide that facilitates secretion of the anti-ghrelin monoclonal antibody light and/or heavy chain from a host cell. The anti-ghrelin monoclonal antibody light and/or heavy chain gene can be cloned into the vector such that the signal peptide is operably linked in-frame to the amino terminus of the antibody chain gene. The signal peptide can be an immunoglobulin signal peptide or a heterologous signal peptide.

In addition to the antibody heavy and/or light chain gene(s), a recombinant expression vector of the invention carries regulatory sequences that control the expression of the antibody chain gene(s) in a host cell. The term “regulatory sequence” is intended to include promoters, enhancers and other expression control elements (e.g., polyadenylation signals), as needed, that control the transcription or translation of the antibody chain gene(s). The design of the expression vector, including the selection of regulatory sequences may depend on such factors as the choice of the host cell to be transformed, the level of expression of protein desired. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma virus.

In addition to the antibody heavy and/or light chain genes and regulatory sequences, the recombinant expression vectors of the invention may carry additional sequences, such as sequences that regulate replication of the vector in host cells (e.g., origins of replication) and one or more selectable marker genes. The selectable marker gene facilitates selection of host cells into which the vector has been introduced. For example, typically the selectable marker gene confers resistance to drugs, such as G418, hygromycin, or methotrexate, on a host cell into which the vector has been introduced. Preferred selectable marker genes include the dihydrofolate reductase (DHFR) gene (for use in DHFR-minus host cells with methotrexate selection/amplification), the neo gene (for G418 selection), and glutamine synthetase (GS) in a GS-negative cell line (such as NS0) for selection/amplification.

For expression of the light and/or heavy chains, the expression vector(s) encoding the heavy and/or light chains is transfected into a host cell by standard techniques e.g., electroporation, calcium phosphate precipitation, DEAE-dextran transfection and the like. Although it is theoretically possible to express the antibodies of the invention in either prokaryotic or eukaryotic host cells, preferably eukaryotic cells, and most preferably mammalian host cells, because such cells, are more likely to assemble and secrete a properly folded and immunologically active antibody. Preferred mammalian host cells for expressing the recombinant antibodies of the invention include Chinese Hamster Ovary (CHO cells) (including DHFR-CHO cells, described in Urlaub and Chasin, Proc. Natl. Acad. Sci. USA 77:4216-20 (1980), used with a DHFR selectable marker, e.g., as described in Kaufman and Sharp, J. Mol. Biol. 159:601-21 (1982)), NS0 myeloma cells, COS cells, and SP2/0 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the host cell and/or the culture medium using standard purification methods.

Host cells can also be used to produce portions, or fragments, of intact antibodies, e.g., Fab fragments or scFv molecules. It will be understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all the DNA encoding either or both of the light and heavy chains that is not necessary for binding to ghrelin. The molecules expressed from such truncated DNA molecules are also encompassed by the antibodies of the invention.

In a preferred system for recombinant expression of an antibody of the invention, a recombinant expression vector encoding both the antibody heavy chain and the antibody light chain is introduced into DHFR-CHO cells by calcium phosphate-mediated transfection. Within the recombinant expression vector, the antibody heavy and light chain genes are each operably linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Antibodies, or antigen-binding portions thereof, of the invention can be expressed in an animal (e.g., a goat) that is transgenic for an antibody of the invention. Plant cells can also be modified to create transgenic plants that express the antibody, or an antigen-binding portion thereof, of the invention.

In view of the foregoing, another embodiment of the invention pertains to nucleic acids, vectors, and host cell compositions that can be used for recombinant expression of the antibodies and antibody portions of the invention. Preferably, the invention provides isolated nucleic acids that encode an anti-hGhrelin monoclonal antibody comprising one or more CDRs with a sequence as shown in SEQ ID Nos: 5, 8, 10, 19, 21 or 25 and even more preferably those CDRs exist in the expressed protein at the same CDR site as they exist as shown in Table 1, 6 or 7 herein. Preferably, the invention provides isolated nucleic acids that encode the heavy chain variable region of an anti-hGhrelin monoclonal antibody comprising an HCVR amino acid sequence as shown in SEQ ID NO: 16 and/or a LCVR with a sequence as shown in SEQ ID NO:3. In one embodiment, a polynucleotide of the invention encoding an anti-hGhrelin monoclonal antibody comprises a polynucleotide comprising a sequence selected from the group consisting of SEQ ID Nos: 1, 2, 4, 6, 7, 9, 11, 12, 13, 14, 15, 17, 18, 20, 22, 23 and 24.

The invention also provides recombinant expression vectors encoding both an antibody heavy chain and/or an antibody light chain. For example, in one embodiment, the invention provides a recombinant expression vector encoding:
a) an antibody heavy chain having a variable region comprising at least one peptide with an amino acid sequence selected from the group consisting of SEQ ID NOs: 19, 21 and 25; and further comprising,
b) an antibody light chain having a variable region comprising at least one peptide with an amino acid sequence selected from the group consisting of SEQ ID NOs: 5, 8 and 10.

The invention also provides host cells into which one or more of the recombinant expression vectors of the invention have been introduced. Preferably, the host cell is a mammalian host cell, more preferably the host cell is a CHO cell, an NS0 cell or a COS cell or a yeast cell. Still further the invention provides a method of synthesizing a recombinant human antibody of the invention by culturing a host cell of the invention in a suitable culture medium until a recombinant humanized antibody of the invention is synthesized. The method can further comprise isolating the recombinant human antibody from the culture medium.

Once expressed, the intact antibodies, their dimers, individual light and heavy chains, or other immunoglobulin forms of the present invention can be purified according to standard procedures of the art, including ammonium sulfate precipitation, ion exchange, affinity, reverse phase, hydrophobic interaction column chromatography, gel electrophoresis and the like. Substantially pure immunoglobulins of at least about 90%, 92%, 94% or 96% homogeneity are preferred, and 98 to 99% or more homogeneity most preferred, for pharmaceutical uses. Once purified, partially or to homogeneity as desired, the peptides may then be used therapeutically or prophylactically, as directed herein.

Chimeric Antibodies

As used herein, the term “chimeric antibody” includes monovalent, divalent or polyvalent immunoglobulins. A monovalent chimeric antibody is a dimer formed by a chimeric heavy chain associated through disulfide bridges with a chimeric light chain. A divalent chimeric antibody is a tetramer formed by two heavy chain-light chain dimers associated through at least one disulfide bridge.

A chimeric heavy chain comprises an antigen-binding region derived from the heavy chain of a non-human antibody specific for ghrelin, which is linked to at least a portion of a human heavy chain constant region, such as CH1 or CH2.

A chimeric light chain comprises an antigen binding region derived from the light chain of a non-human antibody specific for ghrelin, linked to at least a portion of a human light chain constant region (CL).

Antibodies, fragments or derivatives having chimeric heavy chains and light chains of the same or different variable region binding specificity, can also be prepared by appropriate association of the individual polypeptide chains, according to known method steps.

With this approach, hosts expressing chimeric heavy chains are separately cultured from hosts expressing chimeric light chains, and the immunoglobulin chains are separately recovered and then associated. Alternatively, the hosts can be co-cultured and the chains allowed to associate spontaneously in the culture medium, followed by recovery of the assembled immunoglobulin or fragment.

Methods for producing chimeric antibodies are known in the art (see, e.g., U.S. Pat. Nos. 6,284,471; 5,807,715; 4,816,567; and 4,816,397).

In a preferred embodiment, a gene is created which comprises a first DNA segment that encodes at least the antigen-binding region of non-human origin (e.g., that of Fab 1181 or Fab 1621), such as functionally rearranged variable (V) region with joining (J) segment, linked to a second DNA segment encoding at least a part of a human constant (C) region as described un U.S. Pat. No. 6,284,471 (incorporated herein in its entirety).

Humanized Antibodies

Humanized antibodies have at least three potential advantages over non-human and chimeric antibodies for use in human therapy:

1) Because the effector portion is human, it may interact better with the other parts of the human immune system (e.g., destroy the target cells more efficiently by complement-dependent cytotoxicity or antibody-dependent cellular cytotoxicity.

2) The human immune system should not recognize the framework or constant region of the humanized antibody as foreign, and therefore the antibody response against such an injected antibody should be less than against a totally foreign non-human antibody or a partially foreign chimeric antibody.

3) Injected non-human antibodies have been reported to have a half-life in the human circulation much shorter than the half-life of human antibodies. Injected humanized antibodies will have a half-life essentially identical to naturally occurring human antibodies, allowing smaller and less frequent doses to be given.

Humanization may in some instances adversely affect antigen binding. Preferably the humanized anti-hGhrelin monoclonal antibodies of the present invention will possess a binding affinity for acylated hGhrelin of not less than about 50% and more preferably not less than about 30%, and most preferably not less than 5% of the acylated hGhrelin binding affinity of the parent murine antibody, preferably Fab 1111. Preferably, the humanized antibodies of the present invention will bind the same epitope as does Fab 1111 described herein. Such antibodies can be identified based on their ability to compete with Fab 1111 for binding to acylated hGhrelin or to cells expressing acylated hGhrelin. A monoclonal antibody which competes with Fab 1111 for binding acylated hGhrelin is contemplated to fall within the scope of the present invention.

The design of humanized antibodies of the invention may be carried out as follows. In general, the humanized antibodies are produced by obtaining nucleic acid sequences encoding the HCVR and LCVR of an antibody which binds acylated hGhrelin, identifying the CDRs in said HCVR and LCVR, and grafting such CDR-encoding nucleic acid sequences onto selected human framework-encoding nucleic acid sequences. Preferably, the human framework amino acid sequences are selected such that the resulting antibody is likely to be suitable for in vivo administration in humans. This can be determined, e.g., based on previous usage of antibodies containing such human framework sequence. Preferably, the human framework sequence will not itself be significantly immunogenic.

Alternatively, the amino acid sequences of the frameworks for the antibody to be humanized (e.g., Fab 1111) will be compared to those of known human framework sequences the human framework sequences to be used for CDR-grafting will be selected based on their comprising sequences highly similar to those of the parent antibody, e.g., a murine antibody which binds acylated hGhrelin. Numerous human framework sequences have been isolated and their sequences reported in the art. This enhances the likelihood that the resultant CDR-grafted “humanized” antibody, which contains the CDRs of the parent (e.g., murine) antibody grafted onto the selected human frameworks (and possibly also the human constant region) will substantially retain the antigen binding structure and thus retain the binding affinity of the parent antibody. To retain a significant degree of antigen binding affinity, the selected human framework regions will preferably be those that are expected to be suitable for in vivo administration, i.e., not immunogenic. Alternatively the antibody may be humanized using the method described in Foote, J., et al., Proc. Natl. Acad. Sci. U.S.A. 97: 10679-81 (2000).

In either method, the DNA sequence encoding the HCVR and LCVR regions of the preferably murine anti-hGhrelin antibody must be obtained. Methods for cloning nucleic acid sequences encoding immunoglobulins are well known in the art. Such methods may, for example, involve the amplification of the immunoglobulin-encoding sequqences to be cloned using appropriate primers by polymerase chain reaction (PCR). Primers suitable for amplifying immunoglobulin nucleic acid sequences, and specifically murine HCVR and LCVR sequences have been reported in the literature. After such immunoglobulin-encoding sequences have been cloned, they will be sequences by methods well known in the art.

Once the DNA sequences encoding the CDRs and frameworks of the antibody which is to be humanized have been identified, (see e.g., Tables 6 and 7 herein), the amino acid sequences encoding the CDRs are then identified (deduced based on the nucleic acid sequences and the genetic code and by comparison to previous antibody sequences) and the CDR-encoding nucleic acid sequences are grafted onto selected human framework-encoding sequences. This may be accomplished by use of appropriate primers and linkers. Methods for selecting suitable primers and linkers to prive for ligation of desired nucleic acid sequences is well within the ability of one of ordinary skill in the art.

After the CDR-encoding sequences are grafted onto the selected human framework encoding sequences, the resultant DNA sequences encoding the “humanized” variable heavy and variable light sequences are then expressed to produce a humanized Fv or humanized antibody which binds acylated hGhrelin. Typically, the humanized HCVR and LCVR are expressed as part of a whole anti-hGhrelin antibody molecule, i.e., as a fusion protein with human constant domain sequences whose encoding DNA sequences have been obtained from a commercially available library or which have been obtained using, e.g., one of the above described methods for obtaining DNA sequences, or are in the art. However, the HCVR and LCVR sequences can also be expressed in the absence of constant sequences to produce a humanized anti-hGhrelin Fv. Nevertheless, fusion of human constant sequences is potentially desirable because the resultant humanized anti-hGhrelin antibody may possess human effector fuctions.

Methods for synthesizing DNA encoding a protein of known sequence are well known in the art. Using such methods, DNA sequences which encode the subject humanized HCVR and LCVR sequences (with or without constant regions) are synthesized, and then expressed in a vector system suitable for expression of recombinant antibodies. This may be effected in any vector system which provides for the subject humanized HCVR and LCVR sequences to be expressed as a fusion protein with human constant domain sequences and to associate to produce functional (antigen binding) antibodies or antibody fragments.

Human constant domain sequences are well known in the art, and have been reported in the literature. Preferred human constant light chain sequences include the kappa and lambda constant light chain sequences. Preferred human constant heavy chain sequences include human gamma 1, human gamma 2, human gamma 3, human gamma 4, and mutated versions thereof which provide for altered effect or function, e.g., enhanced in vivo half-life, reduced Fc receptor binding, and the like.

In some instances, humanized antibodies produced by grafting CDRs (from an antibody which binds acylated hGhrelin) onto selected human frameworks may provide humanized antibodies having the desired affinity to acylated hGhrelin. However, it may be necessary or desirable to further modify specific residues of the selected human framework in order to enhance antigen binding. Preferably, those framework residues of the parent (e.g., murine) antibody which maintain or affect combining-site structures will be retained. These residues may be identified by X-ray crystallography of the parent antibody or Fab fragment, thereby identifying the three-dimensional structure of the antigen-binding site.

References further describing methods involved in humanizing a mouse antibody that may be used are Queen et al., Proc. Natl. Acad. Sci. USA 88:2869, 1991; U.S. Pat. No. 5,693,761; U.S. Pat. No. 4,816,397; U.S. Pat. No. 5,225,539; computer programs ABMOD and ENCAD as described in Levitt, M., J. Mol. Biol. 168:595-620, 1983

The present invention further embraces variants and the equivalents which are substantially homologous to the humanized antibodies and antibody fragments set forth herein. These are contemplated to contain conservative substitution mutations, i.e., the substitution of one or more amino acids by similar amino acids. For example, conservative substitution refers to the substitution of an amino acid with another within the same genral class, e.g., one acidic amino acid with another acidic amino acid, one basic amino acid with another basic amino acid, or one neutral amino acid by another neutral amino acid. What is intended by a conservative amino acid substitution is well known in the art.

Diagnostic Use

An antibody of the invention may be used to diagnose a disorder or disease associated with the expression of acylated ghrelin. In a similar manner, an antibody of the invention can be used in an assay to monitor ghrelin levels in a subject being treated for a ghrelin associated condition. Diagnostic assays include methods that utilize the antibody of the invention and a label to detect acylated ghrelin in a sample, e.g., in a human body fluid or in a cell or tissue extract. Binding compositions, such as, e.g., antibodies, are used with or without modification, and are labeled by covalent or non-covalent attachment of a reporter molecule.

A variety of conventional protocols for measuring ghrelin, including ELISAs, RIAs, and FACS, are known in the art and provide a basis for diagnosing altered or abnormal levels of ghrelin expression. Normal or standard expression values are established using any art known technique, e.g., by combining a sample comprising a ghrelin polypeptide with, e.g., antibodies under conditions suitable to form a ghrelin:antibody complex. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, betagalactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of a radioactive material include ¹²⁵I, ¹³¹I, ³⁵S, or ³H. (See, e.g., Zola, Monoclonal Antibodies: A Manual of Techniques, CRC Press, Inc. (I1987)).

The amount of a standard complex formed is quantitated by various methods, such as, e.g., photometric means. Amounts of ghrelin polypeptide expressed in subject, control, and samples (e.g., from biopsied tissue) are then compared with the standard values. Deviation between standard and subject values establishes parameters for correlating a particular disorder, state, condition, syndrome, or disease with a certain level of expression (or lack thereof) for a ghrelin polypeptide.

Once the presence of a disorder, -state, condition, syndrome, or disease is established and a treatment protocol is initiated, assays are repeated on a regular basis to monitor the level of ghrelin expression. The results obtained from successive assays are used to show the efficacy of treatment over a period ranging from several days to months. With respect to disorders of cell proliferation (e.g., a cancer), the presence of an abnormal amount of ghrelin (either under- or over expressed) in biopsied tissue or fluid from a subject may indicate a predisposition for the development of a disorder, state, condition, syndrome, or disease of cell proliferation or it may provide a means for detecting such a disorder, state, condition, syndrome, or disease prior to the appearance of actual clinical symptoms. A more definitive initial detection may allow earlier treatment thereby preventing and/or ameliorating further progression of cell proliferation.

Pharmaceutical Composition

An antibody of the present invention can be incorporated into pharmaceutical compositions suitable for administration to a subject. The compounds of the invention may be administered alone or in combination with a pharmaceutically acceptable carrier, diluent, and/or excipients, in single or multiple doses. The pharmaceutical compositions for administration are designed to be appropriate for the selected mode of administration, and pharmaceutically acceptable diluents, carrier, and/or excipients such as dispersing agents, buffers, surfactants, preservatives, solubilizing agents, isotonicity agents, stabilizing agents and the like are used as appropriate. Said compositions are designed in accordance with conventional techniques as in e.g., Remington, The Science and Practice of Pharmacy, 19^{th} Edition, Gennaro, Ed., Mack Publishing Co., Easton, Pa. 1995 which provides a compendium of formulation techniques as are generally known to practitioners.

A pharmaceutical composition comprising an anti-hGhrelin monoclonal antibody of the present invention can be administered to a subject at risk for or exhibiting pathologies associated with obesity or related disorders as described herein using standard administration techniques including oral, intravenous, intraperitoneal, subcutaneous, pulmonary, transdermal, intramuscular, intranasal, buccal, sublingual, or suppository administration.

A pharmaceutical composition of the invention preferably is a “therapeutically effective amount” or a “prophylactically effective amount” of an antibody of the invention. A “therapeutically effective amount” refers to an amount effective, at dosages and for periods of time necessary, to achieve the desired therapeutic result. A therapeutically effective amount of the antibody may vary according to factors such as the disease state, age, sex, and weight of the individual, and the ability of the antibody or antibody portion to elicit a desired response in the individual. A therapeutically effective amount is also one in which any toxic or detrimental effect of the antibody, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A therapeutically-effective amount is at least the minimal dose, but less than a toxic dose, of an active agent which is necessary to impart therapeutic benefit to a subject. Stated another way, a therapeutically-effective amount is an amount which induces, ameliorates or otherwise causes an improvement in the disease or disorder being treated, e.g., the obese state of the mammal, e.g., by decreasing body mass index (BMI).

The route of administration of an antibody of the present invention may be oral, parenteral, by inhalation, or topical. Preferably, the antibodies of the invention can be incorporated into a pharmaceutical composition suitable for parenteral administration. The term parenteral as used herein includes intravenous, intramuscular, subcutaneous, rectal, vaginal, or intraperitoneal administration. Peripheral systemic delivery by intravenous or intraperitoneal or subcutaneous injection is preferred. Suitable vehicles for such injections are straightforward.

The pharmaceutical composition typically must be sterile and stable under the conditions of manufacture and storage in the container provided, including e.g., a sealed vial or syringe. Therefore, pharmaceutical compositions may be sterile filtered after making the formulation, or otherwise made microbiologically acceptable. A typical composition for intravenous infusion could have a volume as much as 250-1000 mL of fluid, such as sterile Ringer's solution, physiological saline, dextrose solution and Hank's solution and a therapeutically effective dose, (e.g., 1 to 100 mg/mL, or more) of antibody concentration. Therapeutic agents of the invention may be frozen or lyophilized for storage and reconstituted in a suitable sterile carrier prior to use. Lyophilization and reconstitution can lead to varying degrees of antibody activity loss (e.g., with conventional immunoglobulins, IgM antibodies tend to have greater activity loss than IgG antibodies). Dosages may have to be adjusted to compensate. Generally, pH between 6 and 8 is preferred.

As is well known in the medical arts, dosages for any one subject depends upon many factors, including the patient's size, body surface area, age, the particular compound to be administered, sex, time and route of administration, general health, and other drugs being administered concurrently. A typical dose can be, for example, in the range of 0.001 to 1000 μg; however, doses below or above this exemplary range are envisioned, especially considering the aforementioned factors. The daily parenteral dosage regimen is about 0.1 μg/kg to about 100 mg/kg of total body weight, preferably from about 0.3 μg/kg to about 10 mg/kg and more preferably from about 1 μg/kg to 1 mg/kg, even more preferably from about 0.5 to 10 mg/kg body weight per day. Progress may be monitored by periodic assessment.

Therapeutic Uses

Ghrelin plays a role in the pathophysiology of obesity and a number of related disorders or diseases. Ghrelin is the first circulating hormone shown to stimulate feeding in humans following systemic administration. One study demonstrated that obese subjects do not demonstrate the decline in plasma ghrelin levels as seen after a meal in lean subjects and may therefore lead to increased food consumption (English, P. et al., J Clin. End. & Metabolism, 87:2984-2987, 2002). Therefore, a pharmaceutical composition comprising an anti-hGhrelin monoclonal antibody of the invention may be used to treat or prevent obesity and/or obesity-related disorders such as NIDDM, Prader-Willi syndrome, impaired satiety, hyperphagia.

Obesity, also called corpulence or fatness, is the excessive accumulation of body fat, usually caused by the consumption of more calories than the body uses. The excess calories are then stored as fat, or adipose tissue. To be overweight, if moderate, is not necessarily to be obese, e.g., in muscular individuals. In general, however, a body weight of a subject that is 20 percent or more over the optimum tends to be associated with obesity. Alternatively, obesity may be defined in terms of Body Mass Index (BMI). Human BMI is defined as the body weight of a human in kilograms divided by the square of the height of that individual in meters. Typically, persons with a BMI of between 25 and 29 are considered overweight and a BMI of 29 or greater is considered obese. This may vary in some persons due to differences in gender or body frame. However, typically BMI of 25 or greater defines the point where the risk of disease increases due to excess weight. Assays for measuring energy expenditure, body composition and weight loss in animals that would be useful for determining effect of an antibody of the invention on an obese subject are known in the art, see e.g., International Patent Publication Number WO 01/87335 (incorporated herein by reference).

Hunger is a desire for food and is normal. Hunger typically occurs when caloric intake is less than caloric expenditure (negative energy balance) and in anticipation of an entrained meal even when the individual is in a positive energy balance. Hyperphagia and impaired satiety are defined as excessive ingestion of food beyond that needed for basic energy requirements. Ingestion may occupy unusual amounts of time. Eating maybe obligatory and disrupt normal activity and can be symptomatic of various disorders. Hyperphagic or impaired satiety conditions may occur in association with central nervous system (CNS) disorders including gangliocytoma of the third ventricle, hypothalmic astrocytoma, Kleine-Levin Syndrome, Froehlich's Syndrome, Parkinson's Disease, genetic disorders including Prader-Willi Syndrome (deletion on the long arm of chromosome 15), psychiatric disorders including anxiety, major depressive disorder, depressive phase of bipolar disorder, seasonal affective disorder, and schizophrenia, psychotropic medication, including delta-9 tetrahydrocannabinol, antidepressants and neuroleptics, may induce hyperphagia. Sleep disorders including sleep apnea is also associated with hyperphagia.

Type II diabetes mellitus, also called non-insulin dependent diabetes mellius (NIDDM), is present in subjects whose insulin their body is still capable of producing is not physiologically effective. An individual can be predisposed to NIDDM by both genetic and environmental factors. Heredity, obesity, and increased age play a major role in the onset of NIDDM. Risk factors include prolonged stress, sedentary lifestyle and certain medications affecting hormonal processes in the body. Eighty percent or more of the people with NIDDM are obese indicating obesity to be a predominant link to the development of NIDDM. An antibody of the invention may also be used to treat or prevent eating disorders including, but not limited to, bulimia, anorexia nervosa, binge eating and metabolic syndrome. An antibody of the invention may be used to treat or prevent cancer or cardiovascular disease.

The use of an anti-hGhrelin monoclonal antibody of the present invention for the treatment of at least one of the aforementioned disorders in which ghrelin activity is detrimental is also contemplated herein. Additionally, the use of an anti-ghrelin monoclonal antibody of the present invention for use in the manufacture of a medicament for the treatment of at least one of the aforementioned disorders in which ghrelin activity is detrimental is contemplated.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, includes administration of a compound of the present invention for treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease or disorder or alleviating symptoms or complications thereof. Treatment may be in conjunction with behavior modification such as limitation of food intake and exercise. Treating obesity therefore includes inhibition of food intake, inhibition of weight gain, and/or inducing weight loss in subjects in need thereof.

A pharmaceutical composition of the invention preferably is a “therapeutically effective amount” or a “prophylactically effective amount” of an antibody of the invention. A “therapeutically effective amount” refers to an amount effective, at dosages and for periods of time necessary, to achieve the desired therapeutic result. A therapeutically effective amount of the antibody may vary according to factors such as the disease state, age, sex, and weight of the individual, and the ability of the antibody or antibody portion to elicit a desired response in the individual. A therapeutically effective amount is also one in which any toxic or detrimental effect of the antibody, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation.

Given their ability to bind to ghrelin from multiple species, antibodies of the invention can be used to detect e.g., rat or human ghrelin peptides (e.g., in a biological sample, such as serum or plasma), using a conventional immunoassay, such as an enzyme linked immunosorbent assays (ELISA), a radioimmunoassay (RIA) or tissue immunohistochemistry. The invention provides a method for detecting ghrelin in a biological sample comprising contacting a biological sample with an antibody, or antibody portion, of the invention and detecting either the antibody (or antibody portion) bound to ghrelin or unbound antibody (or antibody portion), to thereby detect ghrelin in the biological sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, betagalactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of a radioactive material include ¹²⁵I, ¹³¹I, ³⁵S, or ³H.

Ghrelin can be assayed in biological fluids by a competition immunoassay utilizing ghrelin standards labeled with a detectable substance and an unlabeled anti-ghrelin monoclonal antibody. In this assay, the biological sample, the labeled ghrelin standards and the anti-ghrelin monoclonal antibody of the invention are combined and the amount of labeled ghrelin standard bound to the unlabeled antibody is determined. The amount of ghrelin in the sample is inversely proportional to the amount of labeled ghrelin standard bound to the anti-ghrelin monoclonal antibody.

An anti-ghrelin antibody of the present invention may be used in a diagnostic assay for ghrelin levels. Various diagnostic assay techniques known in the art may be used, such as competitive binding assays, direct or indirect ELISA sandwich assays and immunoprecipitation assays conducted in either heterogeneous or homogeneous phases. See, e.g., Zola, Monoclonal Antibodies: A Manual of Techniques, CRC Press, Inc. (1987) pp. 147-158. The antibody used in the assay can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound (such as fluorescein isothiocyanate, rhodamine, or luciferin), or an enzyme (such as alkaline phosphatase, β-galactosidase or horseradish peroxidase). Any method known in the art for conjugating the antibody to the detectable moiety may be employed.

The following examples are offered for illustrative purposes only, and are not intended to limit the scope of the present invention in any way.

### EXAMPLES

### Example 1

### Anti-Ghrelin Fab Synthesis

The CDR and framework sequences disclosed herein have been identified from clones of Fab fragments, which were isolated from antibody libraries generated from an array of antibody RNA created by immunized C57B1/6 mice using Omniclonal™ antibody technology (Biosite®, San Diego, Calif.). The mice were immunized with human ghrelin-Dap3-octanamide as shown in FIG. 3. To improve the immunogenicity of this peptide, keyhole limpet hemoncyanin was conjugated to the peptide through a C-terminal cysteine according to standard methods.

### Example 2

### Competitive ELISA Assay

Anti-hGhrelin Fabs 2291 and 1111 were tested in a competitive ELISA assay, a solution phase assay in which a compound that might compete with an antigen for binding to an antibody is first combined with the antibody in solution phase. Then binding of the antibody to the antigen is measured.

Each well of a 96-well plate was coated with 60 μl BSA-hGhrelin antigen (i.e., BSA conjugated, full-length, acylated human ghrelin, 2 μg/ml in carbonate buffer, pH 9.6). The plate was incubated at 4° C. overnight. The wells were aspirated and washed twice with washing buffer (20 mM Tris-Cl, pH 7.4, 0.15 M NaCl, 0. 1% Tween 20). Compounds (i.e., human ghrelin or ghrelin analogs) were diluted into antibody solution. The antibody solution was mouse-anti-human ghrelin Fab. The ghrelin competitor concentration was varied as listed in Tables 2 and 3 below, but the Fab-concentration was kept constant at 0.1 μg/ml in blocking solution (10 mg/ml BSA in wash buffer). After a 1 hour incubation at room temperature, 50 μl of compound-antibody solution was added to BSA-hGhrelin coated wells in triplicate. The plates were incubated for 1 hour at room temperature. The wells were then washed three times with washing buffer.

Peroxidase-conjugated secondary antibody (50 μl goat anti-mouse kappa HRP (Southern Biotech), diluted 1:2000 in blocking buffer) was added to each well and incubated for 1 hour at room temperature. The wells were then washed 4 times with washing buffer. Fifty microliters of chromogenic substrate (i.e., OPD substrate) was added to each well and allowed to develop at room temperature for 10 minutes. The reaction was stopped by adding 100 μl 1N HCl to each well. The absorbance of the wells was read at 490 nm.

In one iteration of this assay, four different compounds were tested with 0.2 μg/ml, 1.0 μg/ml and 5 μg/ml using Fab 2291. The four compounds were (1) Ghrelin (1-8), which is the first eight amino acids of human ghrelin acylated, via an ester linkage, with an octanoyl group on the serine at position 3; (2) hGhrelin; i.e. full-length human ghrelin acylated with an octanoyl group on the serine at position 3; (3) hGhrelin-DAP3-octanamide; i.e., a full-length human ghrelin modified to have a diamino propionic acid group at position 3 with an octanoyl group attached by means of an amide linkage (see FIG. 3); and (4) octanoic acid. The average absorbance from triplicate wells was determined and is listed below in Table 2. These data demonstrate that Fab 2291 binds to an epitope located within the first 8 amino acids of acylated hGhrelin. Fab 2291 did not bind to octanoic acid in the absence of hGhrelin. However, Fab 2291 did bind comparably to hGhrelin-DAP3-octanamide.

TABLE 2
Fab 2291

| | Compound | |
|---|---|---|
| Compound | Concentration | Ave. OD |
| Ghrelin (1-8) | 0 | 0.9316 |
| | 0.2 μg/ml | 0.3992 |
| | 1 μg/ml | 0.1271 |
| | 5 μg/ml | 0.065 |
| hGhrelin (acylated, 1-28) | 0 | 0.8575 |
| | 0.2 μg/ml | 0.4446 |
| | 1 μg/ml | 0.1809 |
| | 5 μg/ml | 0.0815 |
| hGhrelin-DAP3- | 0 | 0.7051 |
| octanamide | 0.2 μg/ml | 0.2667 |
| | 1 μg/ml | 0.1568 |
| | 5 μg/ml | 0.0713 |
| Octanoic acid | 0 | 0.6134 |
| | 0.2 μg/ml | 0.6405 |
| | 1 μg/ml | 0.7603 |
| | 5 μg/ml | 0.7152 |

In another iteration of the assay, Fab 2291 was tested with the following compounds: (1) hGhrelin; (2) hGhrelin-His9-acylated, i.e., full-length human ghrelin acylated with an octanoyl group at the His residue at position 9; (3) hGhrelin (4-28), human ghrelin lacking the first three amino acids and possessing no acylation group; (4) des-acyl-hGhrelin, i.e., full-length, non-acylated human ghrelin.

The average absorbance from triplicate wells was determined and is listed below in Table 3. These data demonstrate that Fab 2291 does not bind to des-acyl-hGhrelin nor to hGhrelin-His9-acylated. Similar results were obtained using Fab 1111. Overall, the data demonstrated that the epitope to which Fab 2291 and to which Fab 1111 binds is located within the first eight amino acids of acylated hGhrelin and is not present in des-acyl-hGhrelin.

TABLE 3
Fab 2291

| | Compound | |
|---|---|---|
| Compound | Concentration | Ave. OD (490 nm) |
| hGhrelin | 0 | 0.7736 |
| | 0.2 μg/ml | 0.3039 |
| | 1 μg/ml | 0.1483 |
| | 5 μg/ml | 0.063 |
| hGhrelin-His9-acylated | 0 | 0.7439 |
| | 0.2 μg/ml | 0.6067 |
| | 1 μg/ml | 0.69 |
| | 5 μg/ml | 0.702 |
| hGhrelin (4-28) | 0 | 0.7439 |
| | 0.2 μg/ml | 0.5968 |
| | 1 μg/ml | 0.6666 |
| | 5 μg/ml | 0.6781 |
| des-acyl-hGhrelin | 0 | 0.7716 |
| | 0.2 μg/ml | 0.6327 |
| | 1 μg/ml | 0.6628 |
| | 5 μg/ml | 0.7289 |

The in vitro FLPR® Calcium Assay system (Molecular Devices) was used with hamster AV12 cells that had been stably transfected to express the human ghrelin receptor (GHS-R1 a). This assay evaluates changes in intracellular calcium as a means of detecting ghrelin/GHS-R1a binding and signaling in the presense or absence of a Fab of the invention.

AV12 cells were grown in growth media (DMEM/F12 (3:1), 5% fetal bovine serum, 50 μg/ml hygromycin and 50 μg/ml zeocin) to about 50-90×10⁶ cells per T-150 flask. The cells were then trypsinized, washed and distributed into Biocoat black poly-D-lysine coated plates (60,000 cells in 100 μl growth media per well). The cells were incubated for about 20 hours at 37° C. in 5% CO₂. The media was removed from the plate and 150 μl HBSS (Gibco 14025-037) was added to each well and then removed. Then dye was loaded into the cells by adding to each well 50 μl loading buffer [5 μM Fluo-4AM (Molecular Devices), 0.05% Pluronic in FLPR buffer (Hank's Balanced Salt with calcium (Gibco), 0.75% BSA (Gibco) and 10 mM HEPES)]. The plate was further incubated at 37° C. in 5% CO₂ for one hour. The wells were then washed twice with HBSS and 50 μl FLPR buffer was added per well.

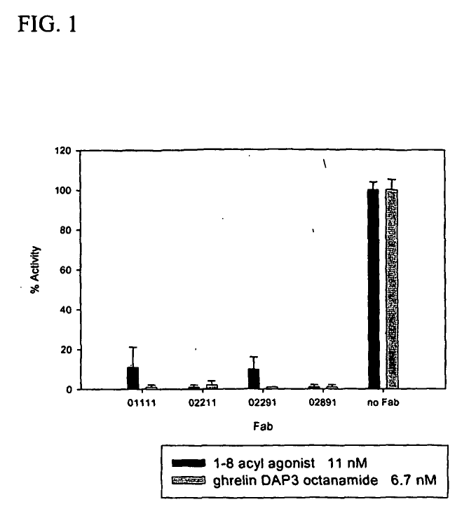

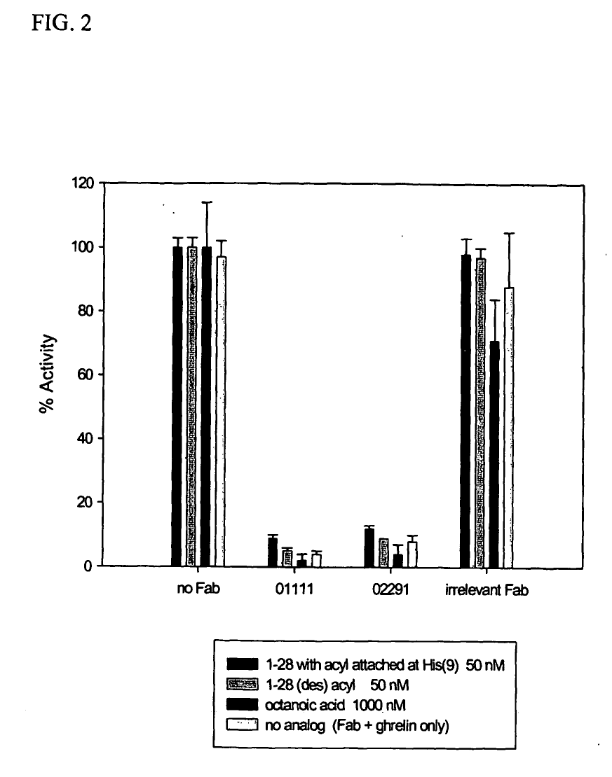

Samples were prepared by combining 7.2 μl calcium concentrate [CaCl₂-2H₂O in water at 3.7 mg/ml mixed 1:1 with HBSS (with calcium) and filter sterilized] with 60 μl sample (Fab of varying concentration), 16.8 μl hGhrelin in 3.75% BSA/50% HBSS. The final concentration of the sample solution was 0.75% BSA, and calcium at approximately the same concentration as in the FLPR buffer). The amount of hGhrelin in the sample is indicated in Tables 4 and 5 and FIGS. 1 and 2. The cell plate was shaken for 15 seconds prior to loading it into the FLPR instrument. Test samples or control samples were added to each well, and read by a Fluorometric Imaging Plate Reader (Molecular Devices).

Active human ghrelin analogs were combined with a Fab of the invention to determine if the Fab could inhibit the analog activity (see FIG. 1). The analogs tested were (1) human ghrelin amino acids 1-8 acylated with octanoic acid attached to the serine at position 3 by an ester linkage and (2) hGhrelin-DAP3-octanamide. These active ghrelin analogs were used at a concentration that yielded sub-maximal activity. The analogs were incubated with the Fab at concentrations known to fully inhibit 1 nM native human ghrelin. Percent activity was calculated as:

$$\frac{Change in fluorescence ( analog + Fab )}{Change in fluorescence ( analog + diluent )} \times 100$$

The data demonstrate that Fabs 1111, 2211, 2291 and 2891 inhibit the activity of both analogs. These four Fabs bound the amino terminal eight amino acids of acylated human ghrelin.

Des-acyl-hGhrelin (non-acylated full-length human ghrelin); hGhrelin-His9-acylated, a weakly active ghrelin analog; and octanoic acid alone, were used to test whether they competed with the full-length, acylated human ghrelin (acylated at Ser3, “acylated ghrelin”) for binding to Fabs 1111, 2211, 2291 and 2891 (see FIG. 2). The molecules competing for Fab binding were used at a concentration at least 50-fold higher than hGhrelin. At this concentration the weak agonist, hGhrelin-His9-acylated, does not show significant activity. The Fab concentration was determined, by titration, to be a level that would give approximately 95% inhibition of ghrelin (1 nM) activity. Percent activity was calculated as stated above. The data demonstrate that, in the absence of an anti-ghrelin Fab, or in the presence of an irrelevant Fab, neither des-acyl-hGhrelin nor hGhrelin-His9-acylated nor octanoic acid alone interfered with hGhrelin activity in the assay. Incubating Fabs 1111, 2211, 2291 and 2891 with acylated ghrelin, in the absence of either des-acyl-hGhrelin, hGhrelin-His9-acylated, or octanoic acid alone resulted in inhibition of the hGhrelin activity. Including either the des-acyl-ghrelin, hGhrelin-His9-acylated, or octanoic acid along with the hGhrelin did not significantly change that inhibition with the Fabs. The data therefore demonstrate that Fabs 1111, 2211, 2291 and 2891 do not bind des-acyl hGhrelin nor octanoic acid alone nor hGhrelin-His9-acylated.

Combining the informnation from these two assay, it is evident that the ghrelin epitope to which Fabs 1111, 2211, 2291 and 2891 bind is localized to human -ghrelin amino acids 1-8 with an acyl group attached to the residue at position 3, (NH2-GSSFLSPE). The epitope is not solely due to the peptide portion nor solely due to the octanoic acid portion of the molecule; both the peptide spanning the eight amino terminal amino acids (1-8) of hGhrelin and the octanoic acid linked to hGhrelin at position 3 must be present to result in the epitope. The epitope requires acylation at the residue at position 3 and not at the His residue at position 9. The four Fabs of the invention that were tested in this assay, although they have different peptide sequences (see SEQ ID NOs: 3 and 16), have the same epitope binding pattern; they all bind to amino acid 1-8 of hGhrelin. Notably, when Fab 1111 was incorporated into a full-length antibody, there was not a statistically significant difference between the ability of the Fab or the full antibody to block hGhrelin activity in the FLPR assay.

TABLE 4
Inhibition of hGhrelin at 0.8 nM

| | |
|---|---|
| Mean Max-Min | Std dev |

Fab 1111 nM

| | | |
|---|---|---|
| 45.2 | 151 | 16 |
| 15 | 141 | 17 |
| 5 | 851 | 39 |
| 1.68 | 2688 | 191 |
| 0.56 | 4801 | 221 |
| 0.186 | 5638 | 219 |
| 0.062 | 6077 | 1423 |
| 0.021 | 6334 | 611 |

Fab 2211 nM

| | | |
|---|---|---|
| 55.95 | 164 | 65 |
| 18.65 | 265 | 48 |
| 6.22 | 1384 | 99 |
| 2.07 | 3133 | 92 |
| 0.69 | 4553 | 159 |
| 0.23 | 5265 | 919 |
| 0.077 | 5634 | 796 |
| 0.026 | 6005 | 1164 |

Fab 2891 nM

| | | |
|---|---|---|
| 21.55 | 102 | 44 |
| 7.18 | 195 | 111 |
| 2.39 | 943 | 105 |
| 0.8 | 2628 | 184 |
| 0.27 | 4023 | 488 |
| 0.09 | 4681 | 579 |
| 0.03 | 5097 | 463 |
| 0.01 | 4946 | 311 |

Fab 2291 nM

| | | |
|---|---|---|
| 23.57 | 130 | 42 |
| 7.86 | 127 | 20 |
| 2.62 | 1080 | 91 |
| 0.87 | 2720 | 230 |
| 0.29 | 4768 | 188 |
| 0.1 | 5019 | 359 |
| 0.011 | 5587 | 503 |

TABLE 5
Inhibition of hGhrelin at 0.8 nM

| Fab | IC50 (nM) |
|---|---|
| 2291 | 0.8 |
| 1111 | 1.1 |
| 2891 | 0.9 |
| 2211 | 1.9 |

### Example 4

### Affinity Measurement of Monoclonal Antibodies

The affinity (K_{D}) of various anti-ghrelin Fabs (1111, 2211, 2291 and 2891) were measured using a BIAcore® 2000 instrument containing a CM5 sensor chip. The BIAcore® utilizes the optical properties of surface plasmon resonance to detect alterations in protein concentration of interacting molecules within a dextran biosensor matrix. Except where noted, all reagents and materials were purchased from BIAcore® AB (Upsala, Sweden). All measurements were performed at 25° C. Samples containing rat or human ghrelin (full length, acylated) were dissolved in HBS-EP buffer (150 mM sodium chloride, 3 mM EDTA, 0.005% (w/v) surfactant P-20, and 10 mM HEPES, pH 7.4). A capture antibody, goat anti-mouse Kappa (Southern Biotechnology, Inc), was immobilized onto flow cells using amine-coupling chemistry. Flow cells (1-4) were activated for 7 minutes with a 1:1 mixture of 0.1 M N-hydroxysuccinimide and 0.1 M 3-(N,N-dimethylamino)propyl-N-ethylcarbodiimide at a flow rate of 10 μl/min. Goat anti-mouse Kappa (30 μg/mL in 10 mM sodium acetate, pH 4.5) was manually injected over all 4 flow cells at a flow rate of 10 μL/min. The surface density was monitored and additional goat anti-mouse Kappa was injected if needed to individual cell until all flow cells reach a surface density of 4500-5000 response units (RU). Surfaces were blocked with a 7 minute injection of 1 M ethanolamine-HCl, pH 8.5(10 μL/min). To ensure complete removal of any noncovalently bound goat anti-mouse Kappa, 15 μL of 10 mM glycine, pH 1.5 was injected twice. Running buffer used for kinetic experiments contained 10 mM HEPES, pH 7.4, 150 mM NaCl, 0.005% P20.

Collection of kinetic binding data was performed at maximum flow rate (100 μL/min) and a low surface density to minimize mass transport effects. Each analysis cycle consisted of (i) capture of 300-350 RU of Fabs(BioSite) by injection of 5-10 μL of 5 μg/ml solution over flow cell 2, 3 and 4 for different Fabs at a flow rate of 10 μL/min., (ii) 200 μL injection (2 min) of hGhrelin (concentration range of 50 nM to 1.56 nM in 2-fold dilution increments) over all 4 flow cells with flow cell 1 as the reference flow cell, (iii) 10 min dissociation (buffer flow), (iv) regeneration of goat anti-mouse Kappa surface with a 15 sec injection of 10 mM glycine, pH 1.5, (v) a 30 sec blank injection of running buffer, and (vi) a 2 min stabilization time before start of next cycle. Signal was monitored as flow cell 2 minus flow cell 1, flow cell 3 minus flow cell 1 and flow cell 4 minus flow cell 1. Samples and a buffer blank were injected in duplicate in a random order. Data were processed using BIAevaluation 3.1 software and data were fit to a 1:1 binding model in CLAMP global analysis software and the averages of three measurements are reported below.

| | | | |
|---|---|---|---|
| Fab | Std Dev | K_{D} (k_{off}/kₒₙ) nM | Std Dev |

Human Ghrelin

| | | | | |
|---|---|---|---|---|
| | kₒₙ | | | |
| 2291 | 1.47 × 10⁶ | 3.34 × 10⁵ | 1.08 | 0.40 |
| 1111 | 1.64 × 10⁶ | 4.10 × 10⁵ | 1.04 | 0.31 |
| 2891 | 1.64 × 10⁶ | 2.52 × 10⁵ | 1.01 | 0.163 |
| 2211 | 1.53 × 10⁶ | 3.27 × 10⁵ | 3.38 | 1.00 |
| | k_{off} | | | |
| 2291 | 1.43 × 10⁻³ | 1.09 × 10⁻⁴ | | |
| 1111 | 1.61 × 10⁻³ | 8.88 × 10⁻⁵ | | |
| 2891 | 1.63 × 10⁻³ | 1.25 × 10⁻⁴ | | |
| 2211 | 4.93 × 10⁻³ | 4.59 × 10⁻⁴ | | |

Rat Ghrelin

| | | | | |
|---|---|---|---|---|
| | kₒₙ | | | |
| 2291 | 1.29 × 10⁶ | 1.58 × 10⁵ | 1.68 | 0.99 |
| 1111 | 1.46 × 10⁶ | 2.28 × 10⁵ | 1.14 | 0.27 |
| 2891 | 1.44 × 10⁶ | 2.51 × 10⁵ | 1.16 | 0.18 |
| 2211 | 1.09 × 10⁶ | 2.44 × 10⁵ | 5.42 | 1.48 |
| | k_{off} | | | |
| 2291 | 2.20 × 10⁻³ | 1.44 × 10⁻³ | | |
| 1111 | 1.61 × 10⁻³ | 8.93 × 10⁻⁵ | | |
| 2891 | 1.64 × 10⁻³ | 6.47 × 10⁻⁵ | | |
| 2211 | 5.63 × 10⁻³ | 5.00 × 10⁻⁴ | | |

TABLE 6
1111 Light chain variable region DNA & amino acid sequence.

| | |
|---|---|
| D L V L T Q S P A S L A V S L | 15 |
| 5′ GACCTTGTGCTGACACAGTCTCCTGCTTCCTTAGCTGTATCTCTG | 45 |
| CDR1 | |
| G Q R A T I S C R A S K S V S | 30 |
| GGGCAGAGGGCCACCATCTCATGTAGGGCCAGCAAAAGTGTCAGT | 90 |
| T S G Y S Y M H W Y Q Q K P G | 45 |
| ACATCTGGCTATAGTTATATGCACTGGTACCAACAGAAACCAGGA | 135 |
| CDR2 | |
| Q P P K L L I Y L A S N L E S | 60 |
| CAGCCACCCAAACTCCTCATCTATCTTGCATCCAACCTAGAATCT | 180 |
| G V P A R F S G S G S G T D F | 75 |
| GGGGTCCCTGCCAGGTTCAGTGGCAGTGGGTCTGGGACAGACTTC | 225 |
| T L N I H P V E E E D A A T Y | 90 |
| ACCCTCAACATCCATCCTGTGGAGGAGGAGGATGCTGCAACCTAT | 270 |
| CDR3 | |
| Y C Q H S R E L P Y T F G A G | 105 |
| TACTGTCAGCACAGTAGGGAGCTTCCGTACACGTTCGGTGCTGGG | 315 |
| T K L E L K R | 112 |
| ACCAAGCTGGAGCTGAAACGG 3′ | 336 |

*anti-ghrelin Fabs 2891, 1481 and 2211 have the same amino acid sequence as 1111
*anti-ghrelin Fab 2291 have the same amino acid sequence as 1111 with the exception that amino acid as position 60 is P in 2291
CDR sequences are in bold type

TABLE 7

1111 Heavy chain variable region DNA &
amino acid sequence

| | |
|---|---|
| * | |
| E I Q L Q Q S G A E L M K P G | 15 |
| GAGATCCAGCTGCAGCAGTCTGGAGCTGAGCTGATGAAGCCTGGG | 45 |
| A S V K L S C K A T G Y I F T | 30 |
| GCCTCAGTGAAGCTTTCCTGCAAGGCTACTGGCTACATATTCACT | 90 |
| CDR1 | |
| G Y W I E W V K Q R P G H G L | 45 |
| GGCTACTCGATAGAGTGGGTAAAGCAGAGGCCTGGACATGGCCTT | 135 |
| CDR2 | |
| E W I G E I L P G S G S T N Y | 60 |
| GAGTGGATTGGAGAGATTTTACCTGGAAGTGGTAGTACTAACTAC | 180 |
| N E K F K G K A T F T A D T S | 75 |
| AATGAGAAGTTCAAGGGCAAGGCCACATTCACTGCAGATACATCC | 225 |
| S N T A N M Q L S S L T T E D | 90 |
| TCCAACACAGCCA1WATGCAACTCAGCAGCCTGACAACTGAGGAC | 270 |
| CDR3 | |
| S A I Y Y C A R Y P Q F R L R | 105 |
| TCTGCCATCTATTACTGTGCAAGATACCCCCAGTTCAGGCTACGA | 315 |
| R E R I A Y W G Q G T L V T V | 120 |
| AGGGAAAGGATTGCTTACTGGGGCCAAGGGACTCTGGTCACTGTC | 360 |
| S V A K T T P P S V Y P L A | 134 |
| TCTGTAGCCAAAACGACACCCCCATCTGTCTATCCACTGGCC | 402 |

| Fab | Position | amino acid |
|---|---|---|
| 2291 | 28 | T (in CDR1) |
| | 37 | A |
| 2891 | 28 | T (in CDR1) |
| 1481 | 28 | T (in CDR1) |
| | 107 | G (in CDR3) |
| | 122 | A |
| 2211 | 13 | R |
| | 28 | T (in CDR1) |
| | 103 | G (in CDR3) |

* Anti-ghrelin Fabs 2291, 2891, 1481 and 2211 have the following amino acid changes when compared to the heavy chain variable region of antibody 1111
CDR sequences are in bold type

TABLE 8

| Seq ID | | | |
|---|---|---|---|
| No. | Antibody | Ab region | Type |
| 1 | 2291 | LCVR | polynucleotide |
| 2 | 1111, 2891, 2211, 1481 | LCVR | polynucleotide |
| 3 | 2291, 1111, 2891, 1481, 2211 | LCVR | amino acid |
| 4 | 2291, 1111, 2891, 1481, 2211 | LCVR CDR1 | polynucleotide |
| 5 | 2291, 1111, 2891, 1481, 2211 | LCVR CDR1 | amino acid |
| 6 | 2291 | LCVR CDR2 | polynucleotide |
| 7 | 1111, 2891, 1481, 2211 | LCVR CDR2 | polynucleotide |
| 8 | 2291, 1111, 2891, 1481, 2211 | LCVR CDR2 | amino acid |
| 9 | 2291, 1111, 2891, 1481, 2211 | LCVR CDR3 | polynucleotide |
| 10 | 2291, 1111, 2891, 1481, 2211 | LCVR CDR3 | amino acid |
| 11 | 2291 | HCVR | polynucleotide |
| 12 | 1111 | HCVR | polynucleotide |
| 13 | 2891 | HCVR | polynucleotide |
| 14 | 1481 | HCVR | polynucleotide |
| 15 | 2211 | HCVR | polynucleotide |
| 16 | 2291, 1111, 2891, 1481, 2211 | HCVR | amino acid |
| 17 | 2291, 2891, 1481, 2211 | HCVR CDR1 | polynucleotide |
| 18 | 1111 | HCVR CDR1 | polynucleotide |
| 19 | 2291, 1111, 2891, 1481, 2211 | HCVR CDR1 | amino acid |
| 20 | 2291, 1111, 2891, 1481, 2211 | HCVR CDR2 | polynucleotide |
| 21 | 2291, 1111, 2891, 1481, 2211 | HCVR CDR2 | amino acid |
| 22 | 2291, 1111, 2891 | HCVR CDR3 | polynucleotide |
| 23 | 1481 | HCVR CDR3 | polynucleotide |
| 24 | 2211 | HCVR CDR3 | polynucleotide |
| 25 | 2291, 1111, 2891, 1481, 2211 | HCVR CDR3 | amino acid |
| 26 | human ghrelin | | amino acid |

TABLE 9
Anti-ghrelin Fab sequences
(2291, 1111, 2891, 1481, 2211)

SEQ ID NO: 1

Polynucleotide sequence encoding 2291 light chain
variable region:

| | |
|---|---|
| 5′ GACCTTGTGCTGACACAGTCTCCTGCTTCCTTAGCTGTATCTCTG | 45 |
| GGGCAGAGGGCCACCATCTCATGTAGGGCCAGCAAAAGTGTCAGT | 90 |
| ACATCTGGCTATAGTTATATGCACTGGTACCAACAGAAACCAGGA | 135 |
| CAGCCACCCAAACTCCTCATCTATCTTCCATCCAACCTAGAACCT | 180 |
| GGGGTCCCTGCCAGGTTCAGTGGCAGTGGGTCTGGGACAGACTTC | 225 |
| ACCCTCAACATCCATCCTGTGGAGGAGGAGGATGCTGCAACCTAT | 270 |
| TACTGTCAGCACAGTAGGGAGCTTCCGTACACGTTCGGTGCTGGG | 315 |
| ACCAAGCTGGAGCTGAAACGG 3′ | 336 |
| SEQ ID NO: 2 | |

Polynucleotide seq encoding 1111, 2891, 2211 and 1481
light chain variable region:

| | |
|---|---|
| 5′ GACCTTGTGCTGACACAGTCTCCTGCTTCCTTAGCTGTATCTCTG | 45 |
| GGGCAGAGGGCCACCATCTCATGTAGGGCCAGCAAAAGTGTCAGT | 90 |
| ACATCTGGCTATAGTTATATGCACTGGTACCAACAGAAACCAGGA | 135 |
| CAGCCACCCAAACTCCTCATCTATCTTGCATCCAACCTAGAATCT | 180 |
| GGGGTCCCTGCCAGGTTCAGTGGCAGTGGGTCTGGGACAGACTTC | 225 |
| ACCCTCAACATCCATCCTGTGGAGGAGGAGGATGCTGCAACCTAT | 270 |
| TACTGTCAGCACAGTAGGGAGCTTCCGTACACGTTCGGTGCTGGG | 315 |
| ACCAAGCTGGAGCTGAAACGG 3′ | 336 |
| SEQ ID NO: 3 | |

2291, 1111, 2891, 1481, 2211 light chain variable region
amino acid sequence:

| | |
|---|---|
| DLVLTQSPASLAVSLGQRATISCRASKSVSTSGYSYMHWYQQKPG | 45 |
| QPPKLLIYLASNLEX60GVPARFSGSGSGTDFTLNIHPVEEEDAA | 90 |
| TYYCQHSRELPYTFGAGTKLELKR | 112 |
| wherein X₆₀ is Ser (S) or Pro (P) | |
| SEQ ID NO: 4 | |

```
Polynucleotide sequence encoding 2291, 1111, 2891, 1481,
2211 light chain CDR1:
5′ AGGGCCAGCAAAAGTGTCAGTACATCTGGCTATAGTTATATGCAC 3′
SEQ ID NO: 5
2291, 1111, 2891, 1481, 2211 light chain variable region
CDR1 amino acid sequence:
      RASKSVSTSGYSYSYMH
SEQ ID NO: 6
Polynucleotide sequence encoding 2291 light chain CDR2:
5′ CTTGCATCCAACCTAGAACCT 3′
SEQ ID NO: 7
Polynucleotide sequence encoding 1111, 2891, 1481, 2211
light chain CDR2:
5′ CTTGCATCCAACCTAGAATCT 3′
SEQ ID NO: 8
2291, 1111, 2891, 1481, 2211 light chain variable region
CDR2 amino acid sequence:
      LASNLEX7 wherein X₇ is Pro (P) or Ser (S)
SEQ ID NO: 9
Polynucleotide sequence encoding 2291, 1111, 2891, 1481,
2211 light chain CDR3:
5′ CAGCACAGTAGGGAGCTTCCGTACACGTTC 3′
SEQ ID NO: 10
2291, 1111, 2891, 1481, 2211 light chain variable region
CDR3 amino acid sequence:
      QHSRELPYT
SEQ ID NO: 11
Polynucleotide encoding 2291 heavy chain variable
region:
```

| | |
|---|---|
| 5′ GAGATCCAGCTGCAGCAGTCTGGAGCTGAGCTGATGAAGCCTGGG | 45 |
| GCCTCAGTGAAGCTTTCCTGCAAGGCTACTGGCTACACATTCACT | 90 |
| GGCTACTGGATAGAGTGGGCAAAGCAGAGGCCTGGACATGGCCTT | 135 |
| GAGTGGATTGGAGAGATTTTACCTGGAAGTGGTAGTACTAACTAC | 180 |
| AATGAGAAGTTCAAGGGCAAGGCCACATTCACTGCAGATACATCC | 225 |
| TCCAACACAGCCAACATGCAACTCAGCAGCCTGACAACTGAGGAC | 270 |
| TCTGCCATCTATTACTGTGCAAGATACCCCCAGTTCAGGCTACGA | 315 |
| AGGGAAAGGATTGCTTACTGGGGCCAAGGGACTCTGGTCACTGTC | 360 |
| TCTGTAGCCAAAACGACACCCCCATCTGTCTATCCACTGGCC 3′ | 402 |
| SEQ ID NO: 12 | |

Polynucleotide encoding 1111 heavy chain variable
region:

| | |
|---|---|
| 5′ GAGATCCAGCTGCAGCAGTCTGGAGCTGAGCTGATGAAGCCTGGG | 45 |
| GCCTCAGTGAAGCTTTCCTGCAAGGCTACTGGCTACATATTCACT | 90 |
| GGCTACTGGATAGAGTGGGTAAAGCAGAGGCCTGGACATGGCCTT | 135 |
| GAGTGGATTGGAGAGATTTTACCTGGAAGTGGTAGTACTAACTAC | 180 |
| AATGAGAAGTTCAAGGGCAAGGCCACATTCACTGCAGATACATCC | 225 |
| TCCAACACAGCCAACATGCAACTCAGCAGCCTGACAACTGAGGAC | 270 |
| TCTGCCATCTATTACTGTGCAAGATACCCCCAGTTCAGGCTACGA | 315 |
| AGGGAAAGGATTGCTTACTGGGGCCAAGGGACTCTGGTCACTGTC | 360 |
| TCTGTAGCCAAAACGACACCCCCATCTGTCTATCCACTGGCC | 402 |
| SEQ ID NO: 13 | |

Polynucleotide sequence encoding 2891 heavy chain
variable region:

| | |
|---|---|
| GAGATCCAGCTGCAGCAGTCTGGAGCTGAGCTGATGAAGCCTGGG | 45 |
| GCCTCAGTGAAGCTTTCCTGCAAGGCTACTGGCTACACATTCACT | 90 |
| GGCTACTGGATAGAGTGGGTAAAGCAGAGGCCTGGACATGGCCTT | 135 |
| GAGTGGATTGGAGAGATTTTACCTGGAAGTGGTAGTACTAACTAC | 180 |
| AATGAGAAGTTCAAGGGCAAGGCCACATTCACTGCAGATACATCC | 225 |
| TCCAACACAGCCAACATGCAACTCAGCAGCCTGACAACTGAGGAC | 270 |
| TCTGCCATCTATTACTGTGCAAGATACCCCCAGTTCAGGCTACGA | 315 |
| AGGGAAAGGATTGCTTACTGGGGCCAAGGGACTCTGGTCACTGTC | 360 |
| TCTGTAGCCAAAACGACACCCCCATCTGTCTATCCACTGGCC | 402 |
| SEQ ID NO: 14 | |
| Polynucleotide sequence encoding 1481 heavy chain | |
| variable region: | |
| 5′ GAGATCCAGCTGCAGCAGTCTGGAGCTGAGCTGATGAAGCCTGGG | 45 |
| GCCTCAGTGAAGCTTTCCTGCAAGGCTACTGGCTACACATTCACT | 90 |
| GGCTACTGGATAGAGTGGGTAAAGCAGAGGCCTGGACATGGCCTT | 135 |
| GAGTGGATTGGAGAGATTTTACCTGGAAGTGGTAGTACTAACTAC | 180 |
| AATGAGAAGTTTAAGGGCAAGGCCACATTCACTCCAGATACATCC | 225 |
| TCCAACACAGCCAACATGCAACTCAGCAGCCTGACAACTGAGGAC | 270 |
| TCTGCCATCTATTACTGTGCAAGATACCCCCAGTTCAGGCTACGA | 315 |
| AGGGGAAGGATTGCTTACTGGGGCCAAGGGACTCTGGTCACTGTC | 360 |
| TCTGCAGCCAAAACGACACCCCCATCTGTCTATCCACTGGCC | 402 |
| SEQ ID NO: 15 | |
| Polynucleotide sequence encoding 2211 heavy chain | |
| variable region: | |
| 5′ GAGATCCAGCTGCAGCAGTCTGGAGCTGAGCTGATGAGGCCTGGG | 45 |
| GCCTCAGTGAAGCTTTCCTGCAAGGCTACTGGCTACACATTCACT | 90 |
| GGCTACTGGATAGAGTGGGTAAAGCAGAGGCCTGGACATGGCCTT | 135 |
| GAGTGGATTGGAGAGATTTTACCTGGAAGTGGTAGTACTAACTAC | 180 |
| AATGAGAAGTTCAAGGGCAAGGCCACATTCACTGCAGATACATCC | 225 |
| TCCAACACAGCCAACATGCAACTCAGCAGCCTGACAACTGAGGAC | 270 |
| TCTGCCATCTATTACTGTCCAAGATACCCCCAGTTCGGGCTACGA | 315 |
| AGGGAAAGGATTGCTTACTGGGGCCAAGGGACTCTGGTCACTCTC | 360 |
| TCTGTAGCCAAAACGACACCCCCATCTGTCTATCCACTGGCC | 402 |
| SEQ ID NO: 16 | |
| 2291, 1111, 2891, 1481, 2211 heavy chain variable | |
| region amino acid sequence: | |
| EIQLQQSGAELMX₁₃PGASVKLSCKATGYX₂₈FTGYWIEWX₃₇KQ | 134 |
| RPGHGLEWIGEILPGSGSTNYNEKFKGKATFTADTSSNTANMQLS | |
| SLTTEDSAIYYCARYPQFX₁₀₃LRRX₁₀₇RIAYWGQGTLVTVSVA | |
| KTTPPSVYPLA | |
| wherein X₁₃ is Arg (R) or Lys (K); | |
| X₂₈ is Ile (I) or Thr (T); | |
| X₃₇ is Ala (A) or Val (V); | |
| X₁₀₃ is Arg (R) or Gly (G); | |
| and | |
| X₁₀₇ is Glu (E) or Gly (C). | |
| SEQ ID NO: 17 | |

```
Polynucleotide sequence encoding 2291, 2891, 1481 and
2211 heavy chain CDR1:
5′ GGCTACACATTCACTGGCTACTGGATAGAG 3′
SEQ ID NO: 18
Polynucleotide sequence encoding 1111 heavy chain CDR1:
5′ GGCTACATATTCACTGGCTACTGGATAGAG 3′
SEQ ID NO: 19
2291, 1111, 2891, 1481 and 2211 heavy chain variable
region CDR1 amino acid sequence:
      GYX₃FTGYWIE wherein X3 is Thr (T) or Ile (I)
SEQ ID NO: 20
Polynucleotide sequence encoding 2291, 1111, 2891, 1481
and 2211 heavy chain CDR2:
5′ GAGATTTTACCTGGAAGTGGTAGTACTAACTACAATGAGAAGTTCAAGGGC
SEQ ID NO: 21
2291, 1111, 2891, 1481 and 2211 heavy chain variable
region CDR2 amino acid sequence:
      EILPGSGSTNYNEKFKG
SEQ ID NO: 22
Polynucleotide sequence encoding 2291, 1111, 2891 heavy
chain CDR3:
5′ TACCCCCAGTTCAGGCTACGAAGGGAAAGGATTGCTTAC 3′
SEQ ID NO: 23
Polynucleotide sequence encoding 1481 heavy chain CDR3:
5′ TACCCCCAGTTCAGGCTACGAAGGGGAAGGATTGCTTAC 3′
SEQ ID NO: 24
Polynucleotide sequence encoding 2211 heavy chain CDR3:
5′ TACCCCCAGTTCGGGCTACGAAGGGAAAGGATTGCTTAC 3′
SEQ ID NO: 25
2291, 1111, 2891, 1481, 2211 heavy chain variable region
CDR3 amino acid sequence:
      YPQFX₅LRRX₉RIAY wherein X₅ is Arg (R) or Gly (G)
      and X₉ is Glu (E) or Gly (G)
SEQ ID NO: 26
Human Ghrelin amino acid sequence:
      NH2-GSSFLSPEHQRVQQRKESKKPPAKLQPX₂₈-COOH
      Where X₂₈ is Arg (R) or absent
```

1. A monoclonal antibody, or antigen-binding portion thereof, against acylated human ghrelin that preferentially binds acylated human ghrelin with respect to unacylated human ghrelin, comprising:
a light chain variable region comprising a peptide with the amino acid sequence shown in SEO ID NO:3, wherein X₆₀ is Ser (S) or Pro (P), and
a heavy chain variable region comprising a peptide with the amino acid sequence shown in SEO ID NO:16. wherein X₁₃ is Arg (R) or Lys (K); X₂₈ is Ile (I) or Thr (T); X₃₇ is Ala (A) or Val (V); X₁₀₃ is Arg (R) or Gly (G); and X₁₀₇ is Glu (E) or Gly (G).

2. The monoclonal antibody or antigen-binding portion thereof of claim 1, wherein said acylated human ghrelin is acylated at the third residue from the amino terminus.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. The monoclonal antibody or antigen-binding portion thereof of claim 1, which comprises a heavy chain constant region selected from the group consisting of IgG1, IgG2, IgG3, IgG4, IgA, IgE, IgM. and IgD.

26. The monoclonal antibody or antigen-binding portion thereof of claim 1, which comprises a kappa or lambda light chain constant region.

27. The monoclonal antibody or antigen-binding portion thereof of claim 1, which is a Fab fragment.

28. The monoclonal antibody or antigen-binding portion thereof of claim 1, which is a F(ab′)₂ fragment.

29. The monoclonal antibody or antigen-binding portion thereof of claim 1, which is a single chain Fv fragment.

30. (canceled)

31. (canceled)

32. (canceled)

33. (canceled)

34. (canceled)

35. (canceled)

36. (canceled)

37. A pharmaceutical compositions comprising said monoclonal antibody or antigen-binding portion thereof of claim 1, and a pharmaceutically acceptable carrier.

38. (canceled)

39. (canceled)

40. (canceled)

41. (canceled)

42. (canceled)

43. An article of manufactures comprising a packaging material and said monoclonal antibody or antigen-binding portion thereof of claim 1 contained within said packaging material, wherein said monoclonal antibody or antigen-binding portion thereof neutralizes acylated ghrelin activity for treatment or prevention of a subject suffering from a disorder in which ghrelin activity is detrimental, and wherein said packaging material comprises a package insert which indicates that said monoclonal antibody or antigen-binding portion thereof neutralizes by preferentially binding acylated ghrelin with respect to unacylated ghrelin.

44. (canceled)

45. (canceled)


SEQUENCE LISTING (26 sequences)

SEQ ID NO: 1 - DNA, 336 bp, mus sp.
  misc_feature: Polynucleotide sequence encoding 2291 light chain variable region
gaccttgtgctgacacagtctcctgcttccttagctgtatctctggggcagagggccaccatctcatgtagggccagcaaaagtgtcagtacatctggctatagttatatgcactggtaccaacagaaaccaggacagccacccaaactcctcatctatcttgcatccaacctagaacctggggtccctgccaggttcagtggcagtgggtctgggacagacttcaccctcaacatccatcctgtggaggaggaggatgctgcaacctattactgtcagcacagtagggagcttccgtacacgttcggtgctgggaccaagctggagctgaaacgg

SEQ ID NO: 2 - DNA, 336 bp, mus sp.
  misc_feature: Polynucleotide sequence encoding 1111, 2891, 2211, and 1481 light chain variable region
gaccttgtgctgacacagtctcctgcttccttagctgtatctctggggcagagggccaccatctcatgtagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtacacgttcggtgctgggaccaagctggagctgaaacgg

SEQ ID NO: 3 - protein, 112 aa, mus sp.
  MISC_FEATURE: 2291, 1111, 2891, 1481, 2211 light chain variable region amino acid sequence
DLVLTQSPASLAVSLGQRATISCRASKSVSTSGYSYMHWYQQKPGQPPKLLIYLASNLEXGVPARFSGSGSGTDFTLNIHPVEEEDAATYYCQHSRELPYTFGAGTKLELKR

SEQ ID NO: 4 - DNA, 45 bp, mus sp.
  misc_feature: Polynucleotide sequence encoding 2291, 1111, 2891, 1481, 2211 light chain CDR1
agggccagcaaaagtgtcagtacatctggctatagttatatgcac

SEQ ID NO: 5 - protein, 17 aa, mus sp.
  MISC_FEATURE: 2291, 1111, 2891, 1481, 2211 light chain variable region CDR1 amino acid
RASKSVSTSGYSYSYMH

SEQ ID NO: 6 - DNA, 21 bp, mus sp.
  misc_feature: Polynucleotide sequence encoding 2291 light chain CDR2
cttgcatccaacctagaacct

SEQ ID NO: 7 - DNA, 21 bp, mus sp.
  misc_feature: Polynucleotide sequence encoding 1111, 2891, 1481, 2211 light chain CDR2
cttgcatccaacctagaatct

SEQ ID NO: 8 - protein, 7 aa, mus sp.
  MISC_FEATURE: 2291, 1111, 2891, 1481, 2211 light chain variable region CDR2 amino acid
LASNLEX

SEQ ID NO: 9 - DNA, 30 bp, mus sp.
  misc_feature: Polynucleotide sequence encoding 2291, 1111, 2891, 1481, 2211 light chain CDR3
cagcacagtagggagcttccgtacacgttc

SEQ ID NO: 10 - protein, 9 aa, mus sp.
  MISC_FEATURE: 2291, 1111, 2891, 1481, 2211 light chain variable region CDR3 amino acid
QHSRELPYT

SEQ ID NO: 11 - DNA, 402 bp, mus sp.
  misc_feature: Polynucleotide encoding 2291 heavy chain variable region
gagatccagctgcagcagtctggagctgagctgatgaagcctggggcctcagtgaagctttcctgcaaggctactggctacacattcactggctactggatagagtgggcaaagcagaggcctggacatggccttgagtggattggagagattttacctggaagtggtagtactaactacaatgagaagttcaagggcaaggccacattcactgcagatacatcctccaacacagccaacatgcaactcagcagcctgacaactgaggactctgccatctattactgtgcaagatacccccagttcaggctacgaagggaaaggattgcttactggggccaagggactctggtcactgtctctgtagccaaaacgacacccccatctgtctatccactggcc

SEQ ID NO: 12 - DNA, 402 bp, mus sp.
  misc_feature: Polynucleotide encoding 1111 heavy chain variable region
gagatccagctgcagcagtctggagctgagctgatgaagcctggggcctcagtgaagctttcctgcaaggctactggctacatattcactggctactggatagagtgggtaaagcagaggcctggacatggccttgagtggattggagagattttacctggaagtggtagtactaactacaatgagaagttcaagggcaaggccacattcactgcagatacatcctccaacacagccaacatgcaactcagcagcctgacaactgaggactctgccatctattactgtgcaagatacccccagttcaggctacgaagggaaaggattgcttactggggccaagggactctggtcactgtctctgtagccaaaacgacacccccatctgtctatccactggcc

SEQ ID NO: 13 - DNA, 402 bp, mus sp.
  misc_feature: Polynucleotide sequence encoding 2891 heavy chain variable region
gagatccagctgcagcagtctggagctgagctgatgaagcctggggcctcagtgaagctttcctgcaaggctactggctacacattcactggctactggatagagtgggtaaagcagaggcctggacatggccttgagtggattggagagattttacctggaagtggtagtactaactacaatgagaagttcaagggcaaggccacattcactgcagatacatcctccaacacagccaacatgcaactcagcagcctgacaactgaggactctgccatctattactgtgcaagatacccccagttcaggctacgaagggaaaggattgcttactggggccaagggactctggtcactgtctctgtagccaaaacgacacccccatctgtctatccactggcc

SEQ ID NO: 14 - DNA, 402 bp, mus sp.
  misc_feature: Polynucleotide sequence encoding 1481 heavy chain variable region
gagatccagctgcagcagtctggagctgagctgatgaagcctggggcctcagtgaagctttcctgcaaggctactggctacacattcactggctactggatagagtgggtaaagcagaggcctggacatggccttgagtggattggagagattttacctggaagtggtagtactaactacaatgagaagtttaagggcaaggccacattcactgcagatacatcctccaacacagccaacatgcaactcagcagcctgacaactgaggactctgccatctattactgtgcaagatacccccagttcaggctacgaaggggaaggattgcttactggggccaagggactctggtcactgtctctgcagccaaaacgacacccccatctgtctatccactggcc

SEQ ID NO: 15 - DNA, 402 bp, mus sp.
  misc_feature: Polynucleotide sequence encoding 2211 heavy chain variable region
gagatccagctgcagcagtctggagctgagctgatgaggcctggggcctcagtgaagctttcctgcaaggctactggctacacattcactggctactggatagagtgggtaaagcagaggcctggacatggccttgagtggattggagagattttacctggaagtggtagtactaactacaatgagaagttcaagggcaaggccacattcactgcagatacatcctccaacacagccaacatgcaactcagcagcctgacaactgaggactctgccatctattactgtgcaagatacccccagttcgggctacgaagggaaaggattgcttactggggccaagggactctggtcactgtctctgtagccaaaacgacacccccatctgtctatccactggcc

SEQ ID NO: 16 - protein, 134 aa, mus sp.
  MISC_FEATURE: 2291, 111, 2891, 1481, 2211 heavy chain variable region amino acid
EIQLQQSGAELMXPGASVKLSCKATGYXFTGYWIEWXKQRPGHGLEWIGEILPGSGSTNYNEKFKGKATFTADTSSNTANMQLSSLTTEDSAIYYCARYPQFXLRRXRIAYWGQGTLVTVSVAKTTPPSVYPLA

SEQ ID NO: 17 - DNA, 30 bp, mus sp.
  misc_feature: Polynucleotide sequence encoding 2291, 2891, 1481 and 2211 heavy chain CDR1
ggctacacattcactggctactggatagag

SEQ ID NO: 18 - DNA, 30 bp, mus sp.
  misc_feature: Polynucleotide sequence encoding 1111 heavy chain CDR1
ggctacatattcactggctactggatagag

SEQ ID NO: 19 - protein, 10 aa, mus sp.
  MISC_FEATURE: 2291, 1111, 2891, 1481 and 2211 heavy chain variable region CDR1 amino acid
GYXFTGYWIE

SEQ ID NO: 20 - DNA, 51 bp, mus sp.
  misc_feature: Polynucleotide sequence encoding 2291, 1111, 2891, 1481 and 2211 heavy chain CDR2
gagattttacctggaagtggtagtactaactacaatgagaagttcaagggc

SEQ ID NO: 21 - protein, 17 aa, mus sp.
  MISC_FEATURE: 2291, 1111, 2891, 1481, and 2211 heavy chain variable region CDR2 amino acid
EILPGSGSTNYNEKFKG

SEQ ID NO: 22 - DNA, 39 bp, mus sp.
  misc_feature: Polynucleotide sequence encoding 2291, 1111, 2891 heavy chain CDR3
tacccccagttcaggctacgaagggaaaggattgcttac

SEQ ID NO: 23 - DNA, 39 bp, mus sp.
  misc_feature: Polynucleotide sequence encoding 1481 heavy chain CDR3
tacccccagttcaggctacgaaggggaaggattgcttac

SEQ ID NO: 24 - DNA, 39 bp, mus sp.
  misc_feature: Polynucleotide \sequence encoding 2211 heavy chain CDR3
tacccccagttcgggctacgaagggaaaggattgcttac

SEQ ID NO: 25 - protein, 13 aa, mus sp.
  MISC_FEATURE: 2291, 1111, 2891, 1481, 2211 heavy chain variable region CDR3 amino acid
YPQFXLRRXRIAY

SEQ ID NO: 26 - protein, 28 aa, homosapiens
  MISC_FEATURE: Human ghrelin amino acid sequence
GSSFLSPEHQRVQQRKESKKPPAKLQPX